(19) United States
(12) Patent Application Publication
(10) Pub. No.: US 2022/0008481 A1
(43) Pub. Date: Jan. 13, 2022

(54) METHOD OF TREATMENT

(71) Applicants: HUDSON INSTITUTE OF MEDICAL RESEARCH, Clayton (AU); MONASH UNIVERSITY, Clayton (AU)

(72) Inventors: Euan Wallace, Clayton (AU); Rebecca Lim, Cranbourne North (AU)

(21) Appl. No.: 17/328,966

(22) Filed: May 24, 2021

Related U.S. Application Data
(63) Continuation-in-part of application No. 15/735,817, filed on Dec. 12, 2017, now Pat. No. 11,123,376.

(30) Foreign Application Priority Data
Jun. 12, 2015 (AU) 2015902214
Apr. 12, 2016 (AU) 2016901349

(51) Int. Cl. A61K 35/50; A61P 11/00; A61P 21/00; A61P 9/00

(52) U.S. Cl. CPC A61K 35/50; A61P 9/00; A61P 21/00; A61P 11/00

(57) ABSTRACT

The present disclosure relates generally to the methods of treatment of mammalian subjects by an enhanced cell-based therapeutic approach in order to facilitate tissue and neuronal repair, regeneration and/or reparation. Medicaments useful in the treatment of mammalian subjects and methods of production of the medicaments are also encompassed by the present disclosure.

### CROSS-REFERENCE TO RELATED APPLICATIONS

This application is a continuation-in-part of U.S. application Ser. No. 15/735,817, filed De. 12, 2017, which claims priority to International Application No. PCT/AU2016/050468, filed Jun. 10, 2016, which claims priority to Australian Provisional Patent Application No. 2015902214, filed on Jun. 12, 2015 and Australian Provisional Patent Application No. 2016901349, filed on Apr. 12, 2016, the entire contents of which are incorporated herein by this reference.

### BACKGROUND

### Technical Field

The present disclosure relates generally to the methods of treatment of mammalian subjects by an enhanced cell-based therapeutic approach in order to facilitate tissue and neuronal repair, regeneration and/or reparation. Medicaments useful in the treatment of mammalian subjects and methods of production of the medicaments are also encompassed by the present disclosure.

### Relevant Technology

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

Modern medicine has been greatly advanced by the identification of chemical therapeutics and biologic agents such as antibiotics. However, many medicaments have multifactorial functions, some influencing off-target physiological effects. Cell-based therapeutics have been proposed as the next pillar of modern medicine (Fishback et al. (2013) Sci Transl Med 5:179 ps 7).

One of the rate limiting factors in cell-based therapies is the potential for inconsistent product. This is highlighted in trials involving stem cells. Mesenchymal stem cells (MSCs), for example, whilst well characterized in the literature and have achieved clinical trial level, generally require serial passaging for use. This can and frequently does adversely impact on when and how the cells can be used.

One approach to counter this problem has been to use banked mesenchymal progenitor cells. This at least avoids the delay between harvest and therapy. However, this introduces a variability in potency of the cells between donors and does not address the issue of the negative impact of serial passaging.

An interim measure to address this issue is to have a “master” cell bank. Again, this does not overcome the inevitable problem that there is a finite number of passages that the cells can undergo before senescence and epigenetic/karyotypic changes occur (Schellenberg et al. (2011) Aging (Albany, N.Y.) 3: 873-888). There is also a risk of immune rejection after repeated doses.

As a case in point, with marked improvements in obstetric surveillance and neonatal care, an increasing number of premature babies survive resulting in an elevation in the prevalence of “diseases of prematurity”. One particularly debilitating condition is bronchopulmonary dysplasia (BPD) which is an incurable chronic lung disease of very preterm infants. It is characterized by maldevelopment and arrest of alveoli and disruption of the pulmonary capillary architecture. BPD is a major case of morbidity and mortality in newborn children. Survivors of BPD are also at serious risk of obstructive respiratory disease in early adulthood (Doyle et al, (2006) Paediatrics 118: 108-113) and of general chronic ill health and cognitive decline (Lodha et al. (2014) PLoSONE: e90843). Whilst mesenchymal stem cells have been proposed as a possible cell-based therapy for BPD, for the reasons outlined above, there are likely batches of cells with widely differing functional effectiveness which, apart from causing emotional stress, may delay other therapeutic choices. The issue of damage to pulmonary capillary architecture is not only confined to human preterm infants. The animal racing industry and in particular the horse racing industry faces the problem of exercise induced pulmonary haemorrhage (EIPH). In some jurisdictions, a horse, for example, which exhibits a nosebleed more than twice after racing is banned for life from further competition. This can result in devastating economic losses. A chemical therapeutic approach to preventing or treating EIPH is likely to cause ethical concerns in terms of performance enchantment and in any event such an approach is unlikely to regenerate burst capillaries.

The beneficial effects of human amnion epithelial cells (hAECs) have been documented (for examples, Hodges et al. (2012) Am J Obstet Gynerol 206: 448e8-448e15; Murphy et al. (2012) Cell Transplant I: 1477-1492; Vosdoganes et al. (2013) Cytotherapy 15:1021-1029; Yawno et al. (2013) Dev Neurosci 35:272-282). However, there is a need to determine their mechanism of action.

It is clear, therefore, that the problem of cell-based therapies needs to be addressed and an alternative strategy is required.

### SUMMARY

In accordance with the present invention a vesicular vehicle for cellular communication is identified as being released from mammalian amnion epithelial cells (AECs). The vesicles, referred to herein as “amniotic exosomes”, are nanometer-sized extracellular vesicles (50-100 nm) derived from late endosomes and released from cell surfaces.

Taught herein is an improved form of mammalian amnion epithelial cell-based therapy. The improvement comprises the use of the nano-sized amniotic exosomes which are released by the epithelial cells and exert reparative effects by activating endogenous repair mechanisms. Amniotic exosomes are shown herein to act directly on immune cells to inter alia reduce T-cell proliferation, increase macrophage phagocytosis, activate stem cells and inhibit collagen production in activated fibroblasts. It is proposed herein that the amniotic exosomes release a profile of exosomal cargo in the form of proteins (e.g. cytokines) and genetic molecules (e.g. miRNA, mRNAs and non-coding RNAs).

The biogenesis of exosomes involves the formation of intraluminal vesicles by the inward budding of the late endosome's limiting membrane. Late endosomes then fuse with the plasma membrane to release the exosomes. Once secreted, exosomes can either be taken up by target cells located in close proximity to the parent cell or travel to distal sites through the circulation. Mechanistically, exosomes operate as complex vectors that contain parental cell material. They can contain proteins and genetic material, which are then transferred to their target cells.

The present invention is predicated, therefore, on the development of an enhanced approach to cell-based therapy. The present disclosure teaches the use of the amniotic exosomes which are released from mammalian amnion epithelial cells and which have immunomodulatory, pro-regenerative and reparative effects. The amniotic exosomes exert an effect on immune cells to reduce T-cell proliferation, increase macrophage phagocytosis and activate endogenous stem cells through the release of beneficial proteomic and genetic molecules such as miRNA, mRNA and non-coding RNAs. The amniotic exosomes are proposed herein to facilitate tissue repair, regeneration and reparation including wound healing, promote cellular maintenance, induce neuronal protection including ameliorating the effects of neurodegeneration and injury and promoting repair and neuroregeneration. The amniotic exosomes, also suppress collagen production in activated fibroblasts. The exosomes are further proposed to promote repair and regeneration following disease or adverse event in the systemic vasculature such as ischemic reperfusion injury or organ damage including ameliorating kidney, liver, pancreas, heart and lung damage as well as the treatment of fibrotic conditions in those organs (e.g. liver or lung fibrosis). Other disease or adverse events that may benefit from the application of amniotic axosomes include stroke, idiopathic pulmonary fibrosis, bronchopulmonary dysplasia and conditions requiring neuronal repair, regeneration and/or reparation. The exosomes are also useful in promoting myelination and hence are proposed to be useful in the treatment of demyelination diseases or disorders such as multiple sclerosis.

The amniotic exosomes have beneficial effects not only in humans but also non-human mammals. Hence, the present invention extends to human and veterinary applications. AECs from human subjects are referred to herein as “hAECs”.

An example of a veterinary application is the treatment of racing animals including horses, racing dogs and camels for exercise induced pulmonary haemorrhage (EIPH).

The amniotic exosomes can be produced in large quantity by culturing mammalian amnion epithelial cells in a bioreactor and isolating the amniotic exosomes from the conditioned culture medium. The epithelial cells can be maintained as an immortalized cell line. The amniotic exosomes can be isolated when required or stored in a lyophilized state.

An innovative feature of the present invention is that it is not necessary to identify a compatible donor of the mammalian amnion epithelial cells in order to use the amniotic exosomes. The exosomes do not induce an adverse immunological reaction. Rather, donors are selected on the basis of gestational stage and/or other characteristics such as health of a neonate or term babies. In an embodiment, however, the amniotic exosomes are derived from hAECs from patients at the terminal end period of a pregnancy. The amnion epithelial cells produce amniotic exosomes which are at least as good at promoting tissue or neuronal repair, regeneration and/or reparation for different physiological conditions as are amnion epithelial cells. However, there is none of the disadvantages of a cell-based therapy. Hence, an aspect of the present invention is donor selection in order to identify amnion epithelial cells which produce amniotic exosomes useful in treating a desired condition. This can lead to the generation of a bank of amnion epithelial cells. A particular batch of cells can then be selected based on the disease or condition to be treated.

Pharmaceutical compositions comprising the amniotic exosomes, therapeutic kits comprising amniotic exosomes and/or reagents for screening for a suitable donor or amnion epithelial cell line and bioreactor kits are also encompassed by the teachings of the present disclosure.

Taught herein is an enhanced or modified form cell-based therapy. Hence, enabled herein is an improved cell-based therapeutic protocol for treating a mammalian including a human subject by the use of amnion epithelial cells the improvement comprising isolating amniotic exosomes from an immortalized amnion epithelial cell line and systemically or locally administering to the subject in need of tissue or neuronal repair, regeneration and/or reparation including promotion of remyelination.

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIGS. 1A and 1B are photographic representations of (FIG. 1A) an electron micrograph of amniotic exosomes showing typical cup-shaped morphology and approximately 100 nm diameter; (FIG. 1B) expression of markers of exosome biogenesis, Alix and TSG101. Alix and TSG101 are exosome biomarkers.

FIGS. 2A and 2B are graphical representations showing that (FIG. 2A) amniotic exosomes inhibit T-cell proliferation similarly to hAEC conditioned media; (FIG. 2B) amniotic exosomes increase macrophage phagocytosis n=3.

FIG. 3 is a graphical representation showing the tissue:airspace ratio is improved by amniotic exosomes in a bronchopulmonary dysplasia (BPD) mouse model.

FIG. 4 is a diagrammatic representation of the experimental time used in Example 3 depicting intra-amniotic LPS injection at E16, injection of exosomes/cells at postnatal day 4 and cull points (crosses).

FIGS. 5A and 5B are graphical representations showing that (FIG. 5A) term exosomes are more immunosuppressive than preterm exosomes; (FIG. 5B) term exosomes are better able to increase macrophage phagocytosis as shown by pHRodo labeling n=3 donors per group.

FIGS. 6A and 6B are graphical representations showing that amniotic exosomes reverse established lung inflammation and fibrosis in a mouse model of bleomycin-induced lung fibrosis. (FIG. 6A: alpha-SMA staining); (FIG. 6B: % Sirius Red). 6-8 month old female C57B16 mice. 10 μg or 50 μg exosomes from term hAECs, administered intranasally 7 days following bleomycin challenge.

FIG. 7 is a graphical representation showing that amniotic exosomes reverse activation of primary human lung fibroblasts in vitro. When cultured in the presence of 5 mg/mL transforming growth factor β, the exosomes decreased protein levels of α-smooth muscle actin within 24 hours.

FIGS. 8A-8C are diagrammatic representations showing that amniotic exosomes contain miRNAs that target cytokine-cytokine receptor signaling pathways. Yellow boxes indicate a target by one or more miRNAs.

FIGS. 9A and 9B are diagrammatic representations showing that amniotic exosomes contain miRNAs that target Wnt signaling pathways. Yellow boxes indicate a target by one or more miRNAs.

FIG. 10 is a diagrammatic representation showing that amniotic exosomes contain miRNAs that target PI3K-Akt signaling pathways. Yellow boxes indicate a target by one or more miRNAs.

FIG. 11 is a diagrammatic representation showing that amniotic exosomes contain miRNAs target TGFβ signaling pathways. Yellow boxes indicate a target by one or more miRNAs.

FIG. 12A is a graphical representation and FIGS. 12B, C and D are photographic representations showing the lung regenerative effects of amniotic exosomes comprising tissue airspace ratio (%) between healthy control hAECs, term exosomes and preterm exosomes. A “term” exosome is an exosome isolated from hAEC at the end of a pregnancy. The “preterm” exosome is isolated prior to pregnancy term.

FIGS. 13A through 13C are photographic representations showing that amniotic exosomes trigger regeneration in the lungs as do hAECs. The dark stain is evidence of elastin-positive tips.

FIG. 14 is a graphical representation showing that amniotic exosomes, but not fibroblast exosomes, trigger an endogenous stem cell response in the lungs. This response is significantly greater than the response induced by hAECs.

FIGS. 15A and 15B show that amniotic exosomes were anti-fibrotic in the liver as evidenced using the Sirius red stain (A) and α-smooth muscle activin (α-SMA) immunohistochemical analysis of histological liver sections CCL4+saline versus CCL4+exosome.

FIG. 16 is a graphical representation showing the differences at the proteomic level between exosomes from term versus preterm hAECs.

FIG. 17 is a graphical representation of a cellular component comparison between hAECs and total mesenchymal stem cells (MSCs), comparing hAEC total exo to total MSC exo (Anderson et al. (2016) Stem cells).

FIG. 18 is a graphical representation of a biological process comparison between hAEC and total MSC (Anderson et al. (2016) supra), comparing hAEC total exo to total MSC exo (Anderson et al. (2016) supra).

FIG. 19 is a timeline of an experimental mouse bronchopulmonary dysplasia (BPD) model.

FIGS. 20A and 20B are characterizations of human amnion epithelial cell derived extracellular vesicle (hAEC-EV). (FIG. 20A) The representative transmission electron microscopy image of isolated hAEC-EVs. Scale bar=100 nm. (FIG. 20B) The representative figure of particle distribution in hAEC-EVs. There was no significant difference in cell viability, protein yield, particle numbers and their mean size between term and preterm hAECs (FIGS. 20C-20F).

FIGS. 21A-21F show EV surface epitopes detected by MACSPlex (n=5). All hAEC-EVs presented EV common tetraspanins CD9 (FIG. 21A) and CD63 (FIG. 21B), surface epitope CD105 (FIG. 21C), and epithelial marker CD326 (FIG. 21D). There were no differences in expression levels of these surface epitopes between term EVs and preterm EVs. Term EVs expressed higher levels of CD142 (FIG. 21E) and CD133 (FIG. 21F) compared to preterm EVs. (*p<0.05).

FIGS. 22A and 22B show data from an in vitro potency assays (n=5). (A) Preterm EVs suppressed T cell proliferation more significantly than term EVs with lower proliferation index. (B) Term EVs improved macrophage phagocytosis more significantly than preterm EVs. (****p<0.0001, *p<0.05).

FIGS. 23A-23G show the tissue-to-air space ratio on PND 7 and 14 (n=6). FIGS. 23A-23E: Representative images for H&E staining. Scale bar=200 μm. FIG. 23F: On PND7, tissue-to-air space ratio was decreased in saline treated injured group compared to control groups. In contrast to hAEC treatment, only term but not preterm EVs improved lung structure. FIG. 23G: On PND14, tissue-to-air space ratio remained lower in saline treated injured group. Term EV treatment mitigated tissue-to-air space ratio and made it comparable to hAEC treatment group and healthy control group. (*p<0.05, **p<0.01, ***p<0.001).

FIGS. 24A-24G show Hart's staining on PND7 and 14 (n=6). FIGS. 24A-24E: Representative images for Hart's staining on PND14. Scale bar=100 μm. FIG. 24F: Term hAEC injection improved secondary septal crest density to control levels. FIG. 24G: Secondary septal crest density was decreased in the saline treated injured group, hAEC and term EV injection normalized it to control levels. (*p<0.05, **p<0.01, ***p<0.001).

FIGS. 25A-25H show changes in inflammatory cytokine levels in mouse lung lysate (n=6). On PND7, IL-1β (FIG. 25A) and TNF-α (FIG. 25B) levels were increased in injured group, and EVs normalized them to control levels, which is comparable to hAEC treatment. On PND14, IL-1β (FIG. 25C) and MIP-2 (FIG. 25D) levels were higher in preterm EV treated group. (FIG. 25E) Levels of RANTES only increased in the hAEC treatment group, but not in the EV group. LIF (FIG. 25F), MCP-1 (FIG. 25G) and GM-CSF (FIG. 25H) levels were increased in the injured group, and both hAECs and term EVs reduced them to control levels. However, levels of LIF and MCP-1 remained higher in the preterm EV treatment group. (*p<0.05, **p<0.01, ***p<0.001).

FIGS. 26A-26E show BASC and AT2 expression in mouse lungs at PND7 and 14 (n=6). (FIG. 26A) Representative image of pro-SPC and CC10 immunohistochemical staining on mouse lung tissues where arrows point to BASCs (scale bars=50 μm). (FIGS. 26B-26C) The average number of BASCs per terminal bronchiole on PND7 (FIG. 26B) and PND14 (FIG. 26C). In contrast to hAEC treatment group, where the average number of BASCs was significantly higher than control group on PND14, it didn't change in EV groups on both time points. (FIG. 26D-26E) The percentage of AT2 cells was significantly increased in term EV treatment group on both PND7 (FIG. 26D) and 14 (FIG. 26E) compared to control group, which is on contrast to the unchanged AT2 cell percentage in both hAEC and preterm EV treatment groups (*p<0.05, **p<0.01, ***p<0.001, ****p<0.0001).

FIGS. 27A-27G show vWF immunohistochemistry in mouse lung tissue at PND 7 and 14 (n=6). FIGS. 27A-27E: Representative images for vWF staining on PND14. Scale bar=100 μm. The average number of vessels per field of view on PND7 (FIG. 27F) and PND14 (FIG. 27G). The average number of vessels with diameter <50 μm (black bars) was decreased in saline treated injured mice, which was restored after hAEC and term EV treatment on both PND7 and 14. There was no difference in the numbers of larger blood vessels (>50 μm, grey bars) between groups. (*p<0.05, ** p<0.01).

FIGS. 28A-28F show α-SMA immunofluorescence in mouse lung tissue at PND14 (n=6). FIGS. 28A-28E: Representative images for α-SMA immunofluorescence in mouse lung tissues at PND14. Scale bar=50 μm. Vessels are indicated with white arrows. FIG. 28F: By PND 14, arterial medial thickness was increased in injured mice. (*p<0.05, **p<0.01, *** p<0.001, **** p<0.0001).

FIGS. 29A-29F show H&E staining of mouse lung tissue at week 6 (n=6). FIGS. 29A-29E: The representative images for H&E staining on mouse lung tissues by week 6. Scale bar=200 μm. FIG. 29F: hAEC treatment improved the tissue-to-air space ratio compared to saline treated injured mice, but remained lower than controls. Term EV treatment improved the tissue-to-air space ratio, making it between hAEC treatment group and saline treated group. (*p<0.05, **p<0.01, **** p<0.0001).

FIGS. 30A-30E show data from an invasive lung function test on 6-week old mice (n=6). (FIGS. 30A-30B) There were no significant differences in the baseline of either respiratory resistance (Rrs, FIG. 30A) or compliance (Crs, FIG. 30B) between groups. (FIGS. 30C-30D) Compared to control group, Rrs was significantly increased (FIG. 30C) and Crs was significantly decreased (FIG. 30D) at the 100 mg/ml methacholine dose in the injured group, but this effect was diminished with hAEC treatment and also improved with term EV treatment groups. (FIG. 30E) Pressure volume loop (PV loop) on week 6. There was a significant upward shift of the PV loop in the injured group compared to the control group. (*p<0.05, **p<0.01, ***p<0.001, ****p<0.0001).

FIGS. 31A-31F show changes in pulmonary artery flow on 6-week old mice (n=6). FIGS. 31A-31E: Representative images of pulmonary artery flow. FIG. 31F: Changes of PAT/PET (pulmonary artery acceleration time/ejection time) ratio. Injury decreased the ratio of PAT/PET, which indicated the development of pulmonary hypertension. Both hAEC treatment and term EV treatment restored the ratio back to control level, however, preterm EV treatment group didn't have significant changes. (**p<0.01).

FIGS. 32A-32F show changes of RVAWT (right ventricle anterior wall thickness) on 6-week old mice (n=6). FIGS. 32A-32E: Representative images of the RVAWT. FIG. 32F: The RVAWT increased in the injured mice, while both hAEC treatment and term EV treatment groups, but not preterm EV group, decreased the wall thickness to control levels. (**p<0.01, ****p<0.0001).

FIGS. 33A-33G show hAEC-derived exosome administration enhances post-stroke recovery in grip function and limits infarct damage, but does not improve forepaw asymmetry or adhesive removal ability 7 d post-stroke. FIG. 33A: Hanging Wire test assessed grip function, as measured as latency to fall (s). FIG. 33B: Cylinder test assessed forelimb asymmetry, measured by percentage of impaired forelimb use during the test (%). FIG. 33C: Adhesive Removal test assessed ability to remove an adhesive from forepaws, measured as time until adhesive removal (s). FIGS. 33D-33G: Representative thionin-stained coronal brain sections and corresponding bar graph depicting infarct damage, measured as volume (mm³), scale bar=1 mm. sham=circles (n=9-14); squares=stroke+vehicle (n=16-19); triangles=stroke+exosomes (n=9-11); **P<0.01 vs Sham, ***P<0.001 vs Sham, ##P<0.01 vs Vehicle, Two-way ANOVA with Tukey's post-hoc test (FIGS. 33A-33C), *P<0.05, ***P<0.001, One-way ANOVA with Tukey's post-hoc test (FIG. 33G). All data presented as mean±SEM.

FIGS. 34A-34L show hAEC-derived exosome administration reduces the infiltration of neutrophils and macrophages, but not microglia, in the ischaemic hemisphere 7 d post-stroke. Representative images and immunohistochemical analyses of (FIGS. 34A-34D) neutrophils, measured as the mean number of MPO⁺ cells within the ischaemic hemisphere, scale bars=50 μm; (FIGS. 34E-H) macrophages, measured as the mean number of F4/80⁺ cells within the ischaemic hemisphere (excluding infarct), scale bars=20 μm and (FIGS. 34I-34L) microglia, measured as the mean number of ionised calcium-binding adaptor molecule 1 (Iba-1)⁺ cells per 200× field of view within the peri-infarct region, scale bars=50 μm. *P<0.05, **P<0.01, ***P<0.001 vs Sham; #P<0.05 vs Vehicle; One-way ANOVA with Tukey's post-hoc test. All data presented as mean±SEM.

FIGS. 35A-35D show hAEC-derived exosome administration does not reduce the infiltration of T or B cells into the ischaemic hemisphere 7 d post-stroke. Representative images and immunohistochemical analyses of (FIGS. 35A-35B) T cells, measured as CD3⁺ cells within the infarct core, peri-infarct region and total hemisphere, scale bars=20 μm and (FIGS. 35C-35D) B cells, measured as number of B220⁺ cells within the infarct core, peri-infarct region and total hemisphere, scale bars=20 μm. *P<0.05, **P<0.01, ***P<0.001 vs sham, One-way ANOVA with Tukey's post-hoc test. All data presented as mean±SEM.

FIGS. 36A-36H show hAEC-derived exosome administration and infiltration of myeloid cells, but not lymphoid cells, into the brain 7 d post-stroke. Flow cytometry was used to quantify: (FIG. 36A) total leukocytes, (FIG. 36B) myeloid cells, (FIG. 36C) macrophages, (FIG. 36D) M2 macrophages, (FIG. 36E) all T cells, (FIG. 36F) CD4⁺ T cells (FIG. 36G) CD8⁺ T cells and (FIG. 36H) B cells. *P<0.05, **P<0.01, ***P<0.001; #P<0.05 vs Vehicle, One-way ANOVA with Tukey's post-hoc test. All data presented as mean±SEM.

FIGS. 37A-37H show hAEC-derived exosome administration attenuates apoptosis and glial scarring in the ischaemic hemisphere 7 d post-stroke. Representative images and immunohistochemical analyses of (FIGS. 37A-37D) apoptosis, measured as the mean number of cleaved-caspase-3⁺ cells within the ischaemic hemisphere, scale bars=50 μm; (FIGS. 37E-37H) reactive astrocytes, measured as the mean number of glial fibrillary acidic protein (GFAP)⁺ astrocytes per 200× field of view within the peri-infarct region; ***P<0.001 vs Sham; #P<0.05 vs Vehicle, One-way ANOVA with Tukey's post-hoc test. All data presented as mean±SEM.

FIGS. 38A-38D show hAEC-derived exosome administration may improve long-term grip function, but does not improve forepaw asymmetry, adhesive removal or infarct damage 28 d post-stroke. Mice were subjected to either sham surgery or photothrombotic stroke surgery followed by administration of either saline or 10 μg is of hAEC-derived exosomes at 1 and 14 d post-stroke. They were functionally assessed over 28 d and culled afterwards for brain tissue analysis. (FIG. 38A) Hanging Wire test assessed grip function, as measured as latency to fall (s), *P<0.05, **P<0.01, ***P<0.001 vs Sham; #P<0.05 vs Vehicle, Two-way ANOVA with Tukey's post-hoc test. (FIG. 38B) Cylinder test assessed forelimb asymmetry, measured by percentage of impaired forelimb use during the test (%), *P<0.05 Vehicle day 3/7 vs Sham, ***P<0.001 both stroke groups day 1 vs Sham, Two-way ANOVA with Tukey's post-hoc test. (FIG. 38C) Adhesive Removal test assessed ability to remove an adhesive, measured as time until adhesive removal (s), *P<0.05 Vehicle day 3 vs Sham, ***P<0.001 all stroke groups day 1 vs Sham, Two-way ANOVA with Tukey's post-hoc test. (FIG. 38D) Bar graph depicting infarct damage in thionin-stained coronal brain sections, measured as volume (mm³), scale bar=1 mm, ***P<0.001, One-way ANOVA with Tukey's post-hoc test. All data presented as mean±SEM; sham=circles (n=8); squares=stroke+vehicle (n=11); triangles=stroke+exosomes (n=8).

FIGS. 39A-39N show hAEC-derived exosome administration provides no further enhancement to stroke-induced regenerative mechanisms but reduces glial scar density 28 d post-stroke. Representative images and immunohistochemical analyses of (FIGS. 39A-39E) doublecortin (DCX), measured as immunoreactive area (mm²) within the peri-infarct region, scale bars=200 μm; (FIGS. 39F-39H) co-localisation of Ki67 with neuronal nuclei (NeuN), measured as number of Ki67⁺ cells and percentage of those cells co-localised with NeuN within the ischaemic hemisphere, scale bars=20 μm; (FIGS. 39I and 39J) von Willebrand factor (vWF), measured as the mean number of vWF⁺ microvessels per 400× field of view within the peri-infarct region, scale bars=20 μm; (FIGS. 39K and 39L) growth-associated protein-43 (GAP-43), measured as GAP-43⁺ growth cones within the infarct core, scale bars=20 μm; (FIGS. 39M and 39N) Glial fibrillary acidic protein (GFAP), measured as the mean number of GFAP⁺ astrocytes per 400× field of view within the peri-infarct region, scale bars=20 μm. *P<0.05, **P<0.01, ***P<0.001 vs Sham: ##P<0.01 vs Vehicle, One-way ANOVA with Tukey's post-hoc test. All data presented as mean±SEM.

FIGS. 40A and 40B show hAEC-derived exosome administration and the effect on body weight or mortality at 7 and 28 d post-stroke.

FIGS. 41A-41F show hAEC-derived exosome administration and the effect on leukocyte and myeloid cell numbers in the blood 7 d post-stroke. Flow cytometry was used to quantify: (FIG. 41A) total leukocytes, (FIG. 41B) myeloid cells, (FIG. 41C) B cells, (FIG. 41D) T cells, (FIG. 41E) CD4⁺ T cells and (FIG. 41F) CD8⁺ T. *P<0.05, One-way ANOVA with Tukey's post-hoc test. All data presented as mean±SEM.

FIGS. 42A-42F show hAEC-derived exosome administration and the effect on immune cell numbers in the spleen 7 d post-stroke. Mice subjected to either sham surgery or photothrombotic stroke surgery followed by administration of either saline or 10 μg of hAEC-derived exosomes at 1 d post-stroke were assessed at 7 d. Flow cytometry was used to quantify: (FIG. 42A) total leukocytes, (FIG. 42B) myeloid cells, (FIG. 42C) B cells, (FIG. 42D) T cells, (FIG. 42E) CD4⁺ T cells and (FIG. 42F) CD8⁺ T. *P<0.05, One-way ANOVA with Tukey's post-hoc test. All data presented as mean±SEM.

FIGS. 43A-43D show hAEC-derived exosome administration and the effect on macrophage polarisation in the brain 7 and 28 d post-stroke. Mice subjected to either sham surgery or photothrombotic stroke surgery followed by administration of either saline or 10 μg of hAEC-derived exosomes at 1 d (and 14 d) post-stroke were assessed at 7 d and 28 d. Immunohistochemical analyses of macrophage polarisation in the infarct and remaining ischaemic hemisphere at (FIGS. 43A-43B) 7 d (FIGS. 43C-43D) 28 d post stroke. *P<0.05, **P<0.01, ***P<0.001, One-way ANOVA with Tukey's post-hoc test. All data presented as mean±SEM. Sample sizes; sham (n=7), stroke vehicle (n=10-13) and stroke exosomes (n=6-7).

FIGS. 44A-44D show the effect of hAEC-EV administration on neuronal repair, regeneration and/or reparation (myelin damage recovery). (FIG. 44A) overview of the experimental design; The arrows indicate the time of intranasal hAEC-EV administration (10 μg/20 μl). (FIG. 44B) Changes in body weight percentage; The body weight was measured 2 times/week during 5-week cuprizone administrations (n=3 mice/naive control and n=6 mice/other groups). ANOVA with Dunnett's test was performed to analyse the difference between the groups. (FIG. 44C) Myelin damage recovery was analysed using FluoroMyelin Red Fluorescent Myelin staining for myelin basic protein (MBP) in different experimental groups (Naive control(i), Control+PBS (ii), Cuprizone+PBS (iii), and Cuprizone+EVs(iv)). (FIG. 44D) Percentage of myelinated area was measured using ImageJ software and graphed using Prism. Statistical significance was evaluated using a two-way ANOVA followed by a Tukey's post hoc test. The p-value <0.05 was considered statistically significant, **p<0.01, ***p<0.001. Values are shown as means+standard deviation (SD); CPZ=cuprizone.

### DETAILED DESCRIPTION

Throughout this specification, unless the context requires otherwise, the word “comprise” or variations such as “comprises” or “comprising”, will be understood to imply the inclusion of a stated element or integer or method step or group of elements or integers or method steps but not the exclusion of any element or integer or method step or group of elements or integers or method steps.

As used in the subject specification, the singular forms “a”, “an” and “the” include plural aspects unless the context clearly dictates otherwise. Thus, for example, reference to “a disease or condition” includes a single disease or condition, as well as two or more diseases or conditions; reference to “an exosome” includes a single exosome, as well as two or more exosomes; reference to “the disclosure” includes a single and multiple aspects taught by the disclosure; and so forth. Aspects taught and enabled herein are encompassed by the term “invention”. A “disease” or “condition” also includes a “disorder”. All such aspects are enabled within the width of the present invention. Any variants and derivatives contemplated herein are encompassed by “forms” of the invention.

The present disclosure teaches an enhanced cell-based therapy to facilitate the treatment of mammalian subjects for a range of diseases and conditions falling generally within the context of repair, regeneration and reparation of cells, tissues, neurological pathways and endocrine pathways. The present disclosure teaches that amniotic exosomes isolated from conditioned medium used to culture mammalian amnion epithelial cells (AECs) have beneficial immunomodulatory physiological and biochemical properties. In essence, the mammalian amniotic exosomes exert an effect on immune cells to reduce T-cell proliferation, increase macrophage phagocytosis and activate endogenous stem cells through the release of proteomic and genetic molecules such as miRNA, mRNA and non-coding RNA. They also suppress collagen production in activated fibroblasts. Importantly, the amniotic exosomes are not immunogenic and hence, allogeneic amniotic exosomes can be used.

The amniotic exosomes also reverse established lung inflammation and lung fibrosis and reverse activation of primary lung fibroblasts. This also applies to fibrosis of other organs such as the liver, pancreas, heart and kidney. In addition, they contain miRNAs, mRNAs and non-coding RNA, that target cytokine-cytokine receptor signaling pathways, Wnt signaling pathways, PI3K-Akt signaling pathways and TGFβ signaling pathways as well as signaling pathways involved in a diverse range of physiological and neurological processes.

The present specification teaches that the mammalian amniotic exosomes induce repair, regeneration and reparation of cells, tissues including organs, neurological pathways, components of the systemic vasculature as well as promoting wound healing. It is proposed herein that the amniotic exosomes facilitate repair, regeneration and reparation of the neuronal tissue, including brain and spinal cord, and promote repair of neuroregenerative conditions, induce reparation of organ damage following trauma, disease or substance abuse, facilitate repair following stroke or other insult to the brain such as traumatic brain injury. The exosomes are proposed to facilitate remyelination in the treatment of a demyelination disease, condition or disorder such as idiopathic pulmonary fibrosis, multiple sclerosis, optic neuritis, Devic's disease, transverse myelitis, acute disseminated encephalomyelitis and adrenoleukodystrophy and adrenomyeloneuropathy. The amniotic exosomes in an embodiment, facilitate repair of lung damage. This is important in the treatment of bronchopulmonary dysplasia (BPD) in human babies. It also has veterinary application in the treatment of exercise induced pulmonary haemorrhage (EIPH) in racing animals such as horses, racing dogs (e.g. greyhounds) and camels.

Accordingly, the present invention enabled herein is a method of treating a mammalian subject, the method comprising the systemic or local administration of mammalian amniotic exosomes derived from allogeneic mammalian amnion epithelial cells from a donor mammal of the same species.

Reference to a “mammalian subject” includes any mammal requiring treatment. In an embodiment the mammalian subject is a human. The term “AEC” means “amniotic epithelial cell”. When from a human, the AECs are designated “hAECs”.

Hence, the present specification in instructional on a method for treating a human subject, the method comprising the systemic or local administration of human amniotic exosomes derived from allogeneic human amnion epithelial cells from a human donor.

In another embodiment, the mammalian subject is a non-human mammal such as but not limited to a horse, cow, sheep, goat, pig, alpaca, llama, dog, cat or camel.

In an embodiment, the mammalian subject is in need of treatment. The term “treatment” encompasses the repair, regeneration or promotion of regeneration and/or reparation of cells, tissues and physiological pathways including neuronal and endocrinal pathways. Examples include but the present invention is not limited to, repair, regeneration and/or reparation of organs including, circulatory vessels, such as capillaries, arteries and veins including such vessels following ischemic-reperfusion injury or stroke, internal and surface wounds, ulcers and scars, neurodegenerative conditions and injury to the brain and spinal cord including traumatic brain injury and spinal cord injury. The exosomes are also proposed for the treatment of organ fibrosis such as fibrotic diseases, conditions or disorders of the lung, liver, heart, kidney and pancreas. The exosomes are also contemplated for use in the treatment of demyelination diseases, conditions or disorders or diseases such as multiple sclerosis, optic neuritis, Devic's disease, transverse myelitis, acute disseminated encephalomyelitis and adrenoleukodystrophy and adrenomyeloneuropathy. The amniotic exosomes are useful in clinical applications to treat a disease or condition as well as a cosmetic agent to promote skin regeneration or scar or wound healing. As described elsewhere herein, amniotic epithelial cell-derived exosomes are effective at treating stroke, idiopathic pulmonary fibrosis and bronchopulmonary dysplasia, and facilitating neuronal repair, regeneration and/or reparation. Thus, in an embodiment disclosed herein, the mammalian subject is treated for stroke, idiopathic pulmonary fibrosis, bronchopulmonary dysplasia, and/or a neurodegenerative condition (e.g., multiple sclerosis). In an embodiment, the amniotic epithelial cell-derived exosomes are term exosomes; that is, exosomes isolated from mammalian AEC at the end of a pregnancy.

Whilst not intending to limit the present invention to any one theory or mode of action, it is proposed herein that the mammalian amniotic exosomes represent a vesicular vehicle for communication from amnion epithelial cells and release proteomic and genetic molecules which provide a cocktail of beneficial molecules to facilitate repair, regeneration and reparation. It is also proposed that the profile of proteomic and genetic molecules will differ depending on the gestational stage of the donor from which the amnion epithelial cells are obtained. Hence, the present specification teaches the creation of a bank of immortalized mammalian amnion epithelial cells from different donors at different gestational stages. Epithelial cells are then selected from the bank based on the disease or condition in the subject to be treated and based on prolife of proteomic and genetic molecules the amniotic exosomes produce. The present specification teaches that depending on the disease or condition to be treated, amniotic exosomes having a particular proteomic and/or genetic prolife may be preferred.

Accordingly, another aspect taught herein is a method of treating a mammalian subject, the method comprising:

i. optionally identifying a donor;

ii. selecting immortalized amnion epithelial cells from the or a donor based on the proteomic and/or genetic profile of amniotic exosomes which are produced by the epithelial cells in culture;

iii. generating conditioned medium from the selected immortalized amnion epithelial cells;

iv. isolating amniotic exosomes from the conditioned medium; and

v. systemically or locally administering the amniotic exosomes to the mammalian subject.

In an embodiment, the mammal subject is a human subject. Hence, another aspect taught herein is a method of treating a human subject, the method comprising:

i. optionally identifying a donor;

ii. selecting immortalized amnion epithelial cells from the or a donor based on the proteomic and/or genetic profile of amniotic exosomes which are produced by the epithelial cells in culture;

iii. generating conditioned medium from the selected immortalized amnion epithelial cells;

iv. isolating amniotic exosomes from the conditioned medium; and

v. systemically or locally administering the amniotic exosomes to the human subject.

In another embodiment, mammalian amniotic exosomes are isolated and their proteomic and genetic profile predetermined and a bank of selected mammalian amniotic exosomes is generated based on the profiles. Particular amniotic exosomes are then selected for use in treatment.

Hence, the present specification is instructional for a method of treating a mammalian subject, the method comprising:

i. optionally identifying a donor;

ii. selecting amniotic exosomes from the or a donor based on the proteomic and/or genetic profile of agents released by the exosomes; and

iii. systemically or locally administering the amniotic exosomes to the mammalian subject.

In an embodiment, the mammalian subject is a human.

Accordingly, taught herein is a method of treating a human subject, the method comprising:

i. optionally identifying a donor;

ii. selecting amniotic exosomes from the or a donor based on the proteomic and/or genetic profile of agents released by the exosomes; and

iii. systemically or locally administering the amniotic exosomes to the human subject.

The amniotic exosomes when used in therapy may also be referred to as a medicament, agent, therapeutic, cell therapy derived agent, active ingredient and the like. Reference to “therapy” includes both clinical and cosmetic therapies.

Further taught herein is a method of inducing cellular or neuronal repair, regeneration and/or reparation in a mammalian subject, the method comprising the systemic or local administration to the mammalian subject of allogeneic amniotic exosomes for a time and under conditions sufficient to induce cellular or neuronal repair.

In an embodiment, a method in enabled herein of inducing cellular or neuronal repair, regeneration and/or reparation in a human subject, the method comprising the systemic or local administration to the human subject of allogeneic amniotic exosomes for a time and under conditions sufficient to induce cellular or neuronal repair.

In a further embodiment, contemplated herein is the use of mammalian amniotic exosomes in the manufacture of a medicament for cellular or neuronal repair, regeneration and/or reparation in a mammalian subject.

In an embodiment, the mammal is a human.

Hence, the present specification further teaches the use of human amniotic exosomes in the manufacture of a medicament for cellular or neuronal repair in a human subject. The present specification further teaches the use of human amniotic exosomes in the manufacture of a medicament for the treatment of a demyelinating disease, condition or disorder such as but not limited to multiple sclerosis.

Taught herein is an isolated sample of amniotic exosomes derived from amnion epithelial cell. This includes an isolated sample of human amniotic exosomes from human amniotic exosomes amnion epithelial cell. It is proposed to use these amniotic exosomes in an improved cell-based therapeutic protocol. The present invention extends, therefore, to a pharmaceutical composition comprising allogeneic mammalian amniotic exosomes selected for use to treat a mammalian subject, the pharmaceutical composition further comprising one or more pharmaceutically acceptable carriers, excipients and/or diluents.

In an embodiment, the mammalian subject is a human subject.

Hence, the present invention teaches a pharmaceutical composition comprising human allogeneic amniotic exosomes for use to treat a human subject, the pharmaceutical composition further comprising one or more pharmaceutically acceptable carriers, excipients and/or diluents.

In addition, the composition may be a cosmetic composition comprising human allogeneic amniotic exosomes for use to treat a human subject, the cosmetic composition further comprising one or more cosmetically acceptable carries, excipients and/or diluents.

Pharmaceutically acceptable carriers can contain a physiologically acceptable compound that acts to, for example, stabilize the amniotic exosomes. Physiologically acceptable compounds can include, for example, carbohydrates, such as glucose, sucrose, or a dextran, antioxidants, such as ascorbic acid or glutathione, chelating agents, low molecular weight proteins, or excipients including water or saline or other stabilizers and/or buffers. Detergents can also used to stabilize or to increase or decrease the absorption of the amniotic exosomes, including liposomal carriers. Pharmaceutically acceptable carriers and formulations are known to the skilled artisan and are described in detail in the scientific and patent literature, see e.g., Remington's Pharmaceutical Sciences (1990), 18^{th} Edition, Mack Publishing Company, Easton, (“Remington's”).

Other physiologically acceptable compounds include preservatives which are useful for preventing the growth or action of microorganisms in an amniotic exosome formulation. Various preservatives are well known and include, e.g., ascorbic acid. One skilled in the art would appreciate that the choice of a pharmaceutically acceptable carrier including a physiologically acceptable compound depends, for example, on the route of administration of the amniotic exosome of the present invention and on the particular physiological or biochemical of the proteins and nucleic acids produced by the exosomes.

Administration of the amniotic exosomes, in the form of a pharmaceutical composition, may be performed by any convenient means known to one skilled in the art and depending on the disease or condition or site of injury. Routes of administration include, but are not limited to, respiratorally, intratracheally, nasopharyngeally, intravenously, intraperitoneally, intrathoracically, subcutaneously, intracranially, intradermally, intramuscularly, intraoccularly, intrathecally, intracereberally, intranasally, rectally, topically, patch, bandage and implant. In an embodiment the amniotic exosomes can be sprayed onto, for example, subject with serious burn wounds.

The pharmaceutical forms suitable for injectable use include sterile aqueous solutions or dispersions.

Sterile injectable solutions in the form of dispersions are generally prepared by incorporating the various sterilized active ingredients into a sterile vehicle which contains the amniotic exosomes.

For parenteral administration, the amniotic exosomes may be formulated with a pharmaceutical carrier and administered as a suspension. Illustrative of suitable carriers are water, saline, dextrose solutions, fructose solutions, ethanol, or oils of animal, vegetative or synthetic origin. The carrier may also contain other ingredients, for example, preservatives, buffers and the like. When the amniotic exosomes are being administered intrathecally, they may also be formulated in cerebrospinal fluid.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g, Sayani and Chien (1996) Crit Rev Ther Drug Carrier Syst 13:85-184.

The amniotic exosomes of the subject invention can also be administered in sustained delivery or sustained release mechanisms, which can deliver the exosomes internally over a period of time. For example, biodegradable microspheres or capsules or other biodegradable polymer configurations capable of sustained delivery of the amniotic exosomes can be included in the formulations of the invention (e.g., Putney and Burke (1998) Nat Biotech 16:153-157).

In preparing pharmaceutical compositions of the present invention, a variety of formulation techniques can be used and manipulated to alter biodistribution. A number of methods for altering biodistribution are known to one of ordinary skill in the art. Examples of such methods include protection of the exosomes in vesicles composed of substances such as proteins, lipids (for example, liposomes), carbohydrates, or synthetic polymers. For a general discussion of pharmacokinetics, see, e.g., Remington's.

The pharmaceutical compositions of the invention can be administered in a variety of unit dosage forms depending upon the method of administration. Such dosages are typically advisorial in nature and are adjusted depending on the particular therapeutic context. The amount of amniotic exosomes adequate to accomplish this is defined as the “effective amount”. The dosage schedule and effective amounts for this use, i.e., the “dosing regimen” will depend upon a variety of factors, including the stage of the disease or condition, the severity of the disease or condition, the general state of the patient's health, the patient's physical status, age, pharmaceutical formulation and concentration or selective of amniotic exosomes. In calculating the dosage regimen for a patient, the mode of administration also is taken into consideration. The dosage regimen must also take into consideration the pharmaceutical composition's rate of clearance, and the like. See, e.g., Remington's; Egleton and Davis (1997), Peptides 18:1431-1439; Langer (1990), Science 249:1527-1533. In an embodiment, from point 0.05 μg to 100 μg of an amniotic exosomes are administered. In this includes from 0.1 μg to 50 μg and 0.1 μg to 20 μg and any amount in between.

In accordance with these methods, the amniotic exosomes or pharmaceutical compositions comprising same may be co-administered in combination with one or more other agents. Reference herein to “co-administered” means simultaneous administration in the same formulation or in two different formulations via the same or different routes or sequential administration by the same or different routes. Reference herein to “sequential” administration is meant a time difference of from seconds, minutes, hours or days between the administration of the amniotic exosomes and another agent. Co-administration may occur in any order. Examples of agents which could be co-administered include cytokines. Generally, the selection of another agent is predicated on the disease or condition to be treated.

Alternatively, targeting therapies may be used to deliver the amniotic exosomes to types of cells or locations in the body, by the use of targeting systems such as antibodies or cell specific ligands. Targeting may be desirable for a variety of reasons, e.g., to promote local treatment at a site in need of treatment.

Further taught herein is the production of amniotic exosomes. Conveniently, this is accomplished in a bioreactor which may be in the form of a batch culture reactor or a continuous flow culture reactor. Generally, the amnion epithelial cells are immortalized and are used to seed growth medium in the bioreactor. The resulting conditioned medium is then collected and the amniotic exosomes isolated and formulated for immediate use or stored such as by lyophilization for later use.

Kits are also contemplated herein. The kits may be therapeutic or diagnostic. The therapeutic kit may comprise a selected batch of lyophilized amniotic exosomes and one or more other pharmaceutically acceptable carriers, excipients and/or diluents and/or another active agent. A diagnostic kit may comprise reagents to determine the proteomic or genetic profile of a batch of amniotic exosomes.

### EXAMPLES

Aspects taught herein are now further described by the following non-limiting Examples.

All data were expressed as mean±standard error of the mean (SEM). All statistical analyses were conducted using Graphpad Prism v 8.3.1 (Graphpad Software Inc., USA). In both studies, all functional testing data were assessed using a Two-Way repeated measures (RM) ANOVA with a Tukey's post-hoc test. All histological and flow cytometrical data were analysed using a One-Way ANOVA with a Tukey's post-hoc test. In all statistical tests, P<0.05 was considered statistically significant.

### Example 1

### Production of Amniotic Exosomes

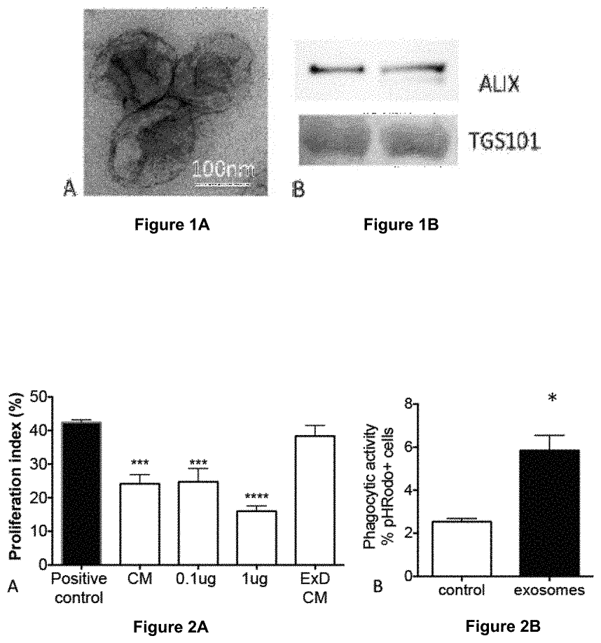

A protocol is developed to isolate amniotic exosomes (FIG. 1). This is the first description of amniotic exosomes and verification of their biological activity. Primary isolates of hAECs are cultured in serum-free media (Ultraculture media, Lonza) for 96 hours before the cells are removed and conditioned media processed for exosome isolation via serial ultracentrifugation at 110,000 g. Approximately 1.5-2 μg purified exosomes per million hAECs are consistently purified regardless of gestational age. This can be scaled up in bioreactor-style cultures without contamination by apoptotic bodies.

The ability of the amniotic exosomes to exert a similar effect was tested. Amniotic exosomes suppress T cell proliferation to a similar extent as hAEC conditioned media, with apparent dose effect (0.1 μg vs 1 μg). Depletion of exosomes from hAEC conditioned media (ExD CM) abolished this effect (FIG. 2A), indicating that amniotic exosomes are a major mediator of T cell suppression. Amniotic exosomes were able to directly increase phagocytic activity of macrophages (FIG. 2B). These findings indicate that the immunomodulatory effects of hAEC conditioned media are largely attributed to exosomes.

### Example 2

### Activity of Amniotic Exosomes

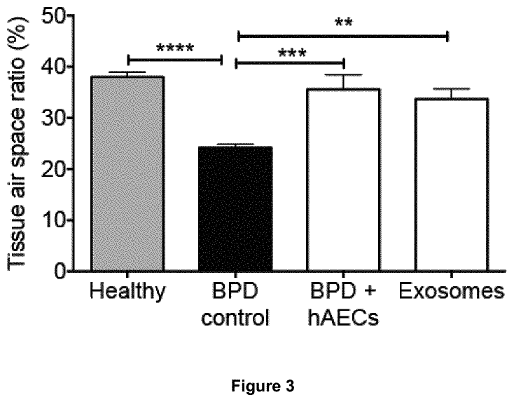

It was determined whether amniotic exosomes were functional in vivo. An aliquot of 1 μg of amniotic exosomes were injected intravenously to BPD mice at postnatal day 4 and an assessment of tissue: airspace ratio performed at postnatal day 14. Amniotic exosomes were effective in reversing alveolar simplification (FIG. 3). Amniotic exosomes play a major role by which they prevent or reverse detrimental changes to lung architecture—by reducing alveolar simplification and recruiting endogenous stem cells, while resolving inflammation in BPD mice.

In summary, the data indicate that amniotic exosomes modulate host immunological events and lung repair in a manner similar to their parent cells. It is proposed that amniotic exosomes can recapitulate the regenerative capabilities of hAECs in vivo. By uncovering the nature of the amniotic exosomal cargo, they can be used to exert a profound immunomodulatory and pro-reparative effect.

A mouse model of BPD is used to determine that neonatal administration of amniotic exosomes can recover lung structure, activate lung stem cell niches and modulate inflammation in BPD mice to levels comparable to that of hAEC treated animals. It is further determined that this will result in improvements in long term physiological outcomes (e.g., pulmonary hypertension and lung function). Proteomic and mRNA/miRNA content of amniotic exosomes are analyzed to identify specific pathways associated with hAEC-mediated repair.

### Example 3

### Reparative Effects of Amniotic Exosomes in BPD Mice

Data indicate that amniotic exosomes exert immunomodulatory and proregenerative effects in vitro and in vivo. To understand how amniotic exosomes affect cellular crosstalk during repair and determined whether amniotic exosomes alone are sufficient to recapitulate the reparative effects of hAECs in an animal model of BPD, the effects of two doses of amniotic exosomes (1 μg and 10 μg) are compared against an optimized dose of hAECs. Fibroblasts and fibroblast exosomes are used as controls.

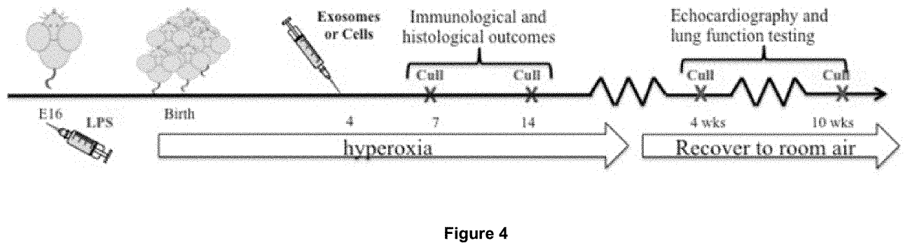

A mouse model of BPD is used, which combines two major contributing factors to human BPD—perinatal inflammation and postnatal hyperoxia—to assess the effects of term and preterm amniotic exosomes on lung repair. While there are limitations to modeling a complex disease like BPD using rodents, this model lends itself to detailed molecular analysis. Rodent studies allow relative affordability for the assessment of dose effects, and long term studies looking into adolescent and adult outcomes. Briefly, 0.2 μg lipopolysaccharide (LPS) in 5 μL saline is injected into each amniotic sac of mouse fetuses at E16 using microforged glass needles (internal diameter: 70-80 μm) and a microinjector (IM-300, Narashige). Once born, newborn mouse pups and their nursing dams are placed into either a hyperoxia chamber (65% oxygen) or room air. Nursing dams are rotated every 48 hours to prevent oxygen toxicity. This combination of prenatal inflammation and postnatal hyperoxia causes lung injury resembling human BPD (Vosdoganes et al. (2013) Cytotherapy 15:1021-1029; Nold et al. Proc. Natl Acad. Sci USA 110:14384-14389) Therapy is administered on postnatal day 4. The experimental timeline depicting intra-amniotic LPS injection at E16, injections of exosomes/cells at postnatal day 4 and cull points (crosses) is shown in FIG. 4.

Exosomes or cells are administered intravenously through the superficial temporal vein, using the same equipment described for intra-amniotic injections and wider glass needles (100-120 μm internal diameter). The final injection volume is 10 μL, which is well tolerated by 4-day old mice. Mouse pups are culled at postnatal days 7 and 14 for assessment of immunological changes and lung stem cell recruitment and lung repair. Two cohorts of animals are then transferred into room air after weaning and tested at 4- and 10-weeks of age to assess the effects of neonatal therapy on long term outcomes, e.g., pulmonary hypertension, cardiovascular and respiratory function during adolescence and early adulthood.

hAECs are isolated from term (37-40 weeks) human pregnancies. Primary isolates are used for the experiments. hAECs from six donors are equally pooled to provide a uniform population for all animal experiments. Animals receiving hAECs receive a single injection of 100,000 hAECs on postnatal day 4. For amniotic exosomes, a portion of the pooled hAECs is placed into culture media (10 million per 25 mL Ultraculture media, Lonza) for 96 hours. Exosomes are then isolated from the conditioned media. The exosomal nature of the isolated pellet by performing western blots for exosomal markers (TSG101 and Alix) as well as size and discrimination by electron microscopy. Exosomes are resuspended in saline and administered at a dose of either in 1 μg or 10 μg at postnatal day 4.

Human lung fibroblasts do not support lung repair and are suitability as a control cell type (Moodley et al. (2010) Am J Respir Crit Care Med i:643-651). Human lung fibroblasts or fibroblast exosomes obtained using the same culture protocol as above are administered. Fibroblasts are administered at the same dosage as hAECs and fibroblast exosomes at the higher dosage (10 μg). Experimental groups are described in the Table 1.

TABLE 1
Experimental Groups

| Healthy mice | | | Group | BPD mice | | | Group |
|---|---|---|---|---|---|---|---|
| Saline | — | | 1 | Saline | — | | 7 |
| hAECs | 100,000 | cells | 2 | hAECs | 100,000 | cells | 8 |
| Amniotic exosomes | 1 | μg | 3 | Amniotic exosomes | 1 | μg | 9 |
| | 10 | μg | 4 | | 10 | μg | 10 |
| Fibroblasts | 100,000 | cells | 5 | Fibroblasts | 100,000 | cells | 11 |
| Fibroblast exosomes | 10 | μg | 6 | Fibroblast exosomes | 10 | μg | 12 |

Immunological Changes

Lungs are collected and processed for flow cytometry as previously described (Nold et al. (2013) supra; Tan et al. (2015) Stem Cell Res. Ther. 6:8). The CD45+fraction is sorted and a combination of surface markers and intracellular cytokine stains used to assess changes to numbers, phenotypes and activation states of T-cells (CD3, CD4, CD25, IFNγ, IL-4, IL17A, FoxP3), macrophages (CD11b, F4/80, CD86, MHCII), neutrophils (CD11c, Ly6G), B cells (B220) and NK cells (NK1.1). Bronchoaloveolar lavage fluid is collected to measure changes in cytokines using a Proteome Profiler (R&D Systems) as previously described (Nold et al. (2013) supra).

### Lung Stem/Progenitor Cell Recruitment

Changes to the BASC population are determined by flow sorting based on the criteria CD45−/CD31−/Sca−1+/EpCam+ (Lee et al. (2014) Cell 156: 440-455). This uses the CD45+ fraction of cells from the immune cell study above. AT2 is sorted based on flow sorting of CD31−/Sca−1−/autofluorescent^{high}. Differences in transcriptional profiles is determined using single cell digital PCR (Fluidigm, qdPCR 37K). Flow sorted single cells are captured on a 96-well microfluidic plate (C1 Single Cell Autoprep System, Fluidigm) where cell lysis, RNA isolation, pre-amplification and cDNA conversion will occur. The samples are then loaded onto microfluidic cards for digital PCR. Data are analyzed using the SINGuLAR v2.0 analysis toolset. Since niche activation pathways of BASC and AT2 are poorly described, a customized 48:48 deltaGene assay that covers stem cell pluripotency, activation, recruitment and differentiation, including the recently described BMP1/NFATc1/Thrombospondin-1 axis (Lee etal. (2014) supra).

### Alveolar Simplification

Quantitative image analysis measuring tissue:airspace ratio is preformed to determine the extent of alveolar simplification across all experimental groups.

### Activation of Host Stem Cell Niche

Immunohistochemical staining (SPC+CC10+) is performed for BASCs at the terminal bronchioles to determine activation states of lung stem cell niches (Lee et al. (2014) supra).

The aim is to know if changes to lung structure and recruitment of endogenous lung stem cells extend to long term improvements in lung function and reduced secondary complications.

### Physiological Studies

Lung function testing and echocardiography is performed on recovered adolescent (4-week old) and young adult (10-week old) mice.

### Echocardiography

The mice are anaesthetized with 3% isoflurane and continued at 1-2% to achieve a heart rate of 350-450 bpm. The Vevo 2100 ultrasound (Monash Bioimaging) and a 40 MHz linear transducer are used to perform PW doppler measurements of pulmonary artery acceleration time along the anteriorly angulated left parasternal long axis view. Right ventricular wall thickness is measured by applying the M mode along the right parasternal long axis view. The same groups of mice are used for invasive lung function testing. They are tracheostomized with an 18G cannula connected to an inline ultrasonic nebuliser, ventilator and attached pressure transducer (FlexiVent, SCIREQ, Montreal, Canada). Airway resistance and compliance are assessed by exposing the mice to increasing concentrations of methacholine (1-30 mg/mL, 3 mins per cycle). Forced expired volumes, vital capacity and inspiratory capacity are obtained. Unlike unrestrained whole body plethysmography, this does not require training of animals and enables a brief pause in mechanical ventilation to execute measurement maneuvers during which predefined pressures or volume waveforms are measured. This overcomes traditional challenges faced in plethysmography such as excessive dead space and measurement inaccuracies.

It is proposed that amniotic exosomes will have a beneficial effect in their ability to trigger macrophage polarization, induce Treg expansion, and reduce activation of neutrophils and dendritic cells in BPD mice. Immunological changes are proposed to be more profound with the 10 μg dose of amniotic exosomes compared to 1 μg of control hAECS. As such, reversal of alveolar simplification is greater in the animals that receive the higher dose of amniotic exosomes. This translates to improvements in long term physiological outcomes such that there will be dose-dependent reduction in right ventricular wall thickening, amelioration of pulmonary hypertension and restoration of normal lung function. No changes are expected when hAECs or amniotic exosomes are given to healthy mice. Fibroblasts or fibroblast exosomes are not proposed to have an effect on immune cells, lung repair or long term physiological outcomes.

### Example 4

### Unique Mediators in Amniotic Exosomes

The gestational age of the hAEC donor can have significant impact on their reparative capacity (Lim et al. (2013) Placenta 34: 486-492). A comparison is made between exosomal cargo collected from term and preterm hAECs. In preparation, amniotic exosomes from term and preterm donors are administered and showed that alveolar simplification is only reversed in animals that received the amniotic exosomes from term donors, thus indicating that the ability to activate pathways for immune modulation and regeneration are significantly impaired in preterm amniotic exosomes. When an initial presence/absence proteomic analysis is performed on the exosomal cargo, 242 and 21 unique proteins in the term and preterm donor, respectively are identified. Using gene ontology analysis, it is determined that term amniotic exosomes contained mediators of cell signaling associated with wound healing, apoptosis, vascular development, acute inflammation and epithelial cell development.

For proteomic analysis, an in-solution trypsin digest of amniotic exosomes (term and preterm, n=10 per group) is performed followed by liquid chromatography and mass spectrometry for absolute quantitation (WEHI Proteomics Laboratory, Melbourne, Australia). Data are acquired using a Q-Exactive hybrid quadrupole-orbitrap mass spectrometer fitted to a Nano-ESI source (Proxeon) coupled to a nanoACQUITY UPLC system (Waters). Peak lists are merged for each LC-MS/MS run into a single MASCOT file and search against a human Ref-Seq protein database (1% false discovery rate). Pipeline Pilot (Accelrys) and Spotfire (TIBCO) is used to analyze quantitative proteomics data. Wilcoxon signed-rank test is used to evaluate differences in abundance. The UniProt database issued to classify proteins based on function, subcellular localization, and specify genes involved in wound healing, cell survival and immune modulation.

For nucleic acid analysis, digital gene expression profiling is performed using Massive Analysis of cDNA ends (MACE, GenXpro GmbH). This allows the capture and quantification rare of transcripts at ˜20 times deeper than RNASeq (1-20 copies per million transcripts) such as receptors and transcription factors, which are usually lost in microarrays. MACE is optimized to sequence small RNA and miRNA from exosomes and combines the benefits of qPCR arrays and RNASeq by tagging each cDNA molecule. It identifies alternative polyadenylation, which influences mRNA-miRNA interaction and thus determines stability and biological relevance of transcripts. Gene ontology enrichment and gene set enrichment analysis for pairwise comparisons are preformed.

There will be unique molecular signatures between term and preterm amniotic exosomes, which relate to their pro-reparative and regenerative effects.

### Example 5

### Pro-Regenerative Effects

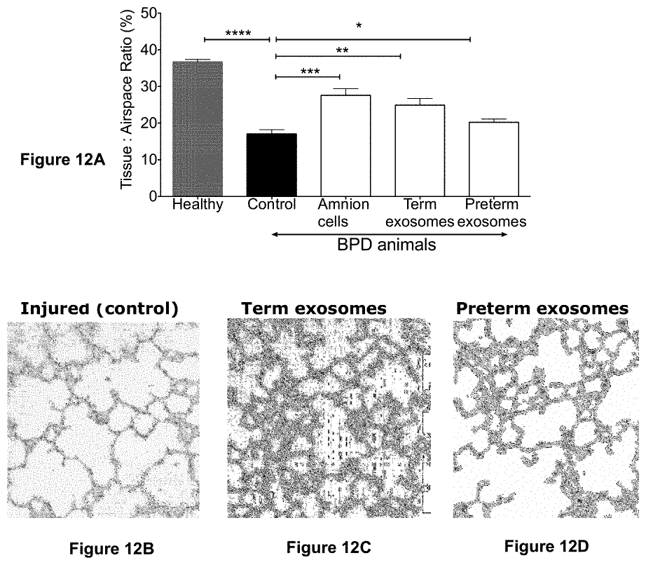

The pro-regenerative effects of amniotic exosomes was demonstrated in a neonatal mouse model of bronchopulmonary dysplasia. Alveolar pruning was observed following the administration of exosomes from term or preterm amniotic tissue (FIGS. 12A through D). The term “BPD” means the bronchopulmonary dysplasia mouse model animals.

### Example 6

### Mechanism of Action of Exosomes

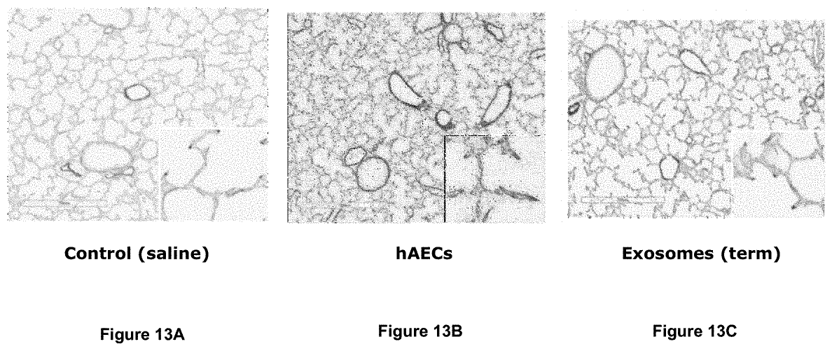

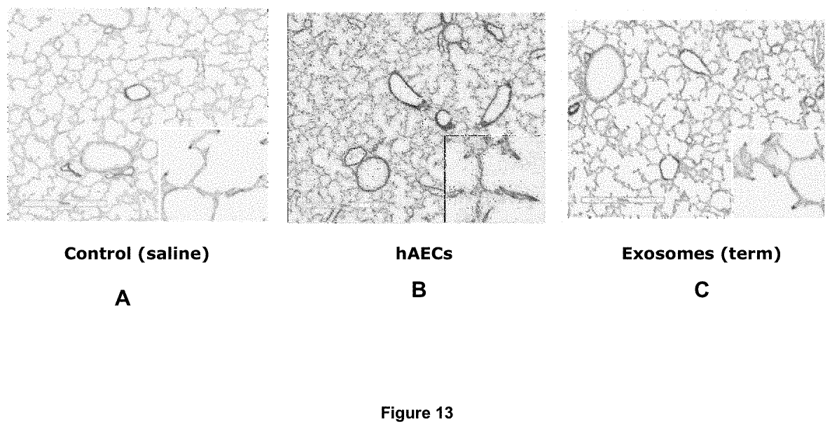

Term-derived human exosomes were tested along side human amniotic epithelial cells (hAECs) for ability to induce lung regeneration. The results are shown in FIGS. 13A through C. Term exosomes restored secondary septal crests as seen as dark stained (elastin positive) tips in FIG. 13.

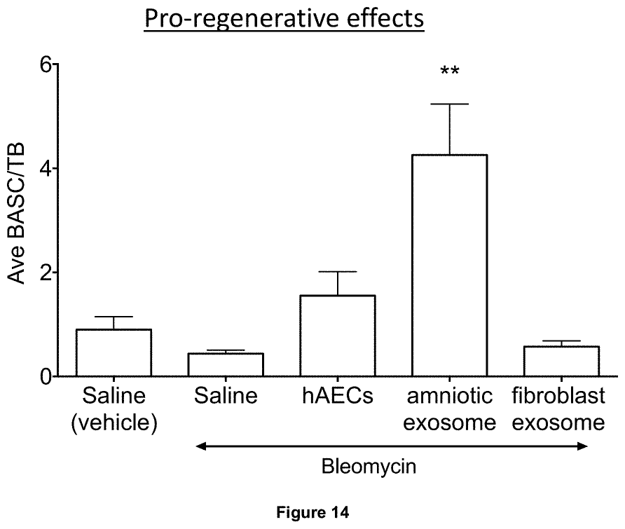

In addition, FIG. 14 shows that amniotic exosomes trigger an endogenous stem cell response in the lungs. In fact, amniotic exosomes were more than twice as effective as were hAECs.

Also observed was that amniotic exosomes could directly stimulate enhancement in the growth of exogenous lung stem cells. This occurred in alveolar, bronchiolar and mixed lung tissue exposed to exosomes, relative to a control.

### Example 7

### Exosomes are Anti-Fibrotic in Liver

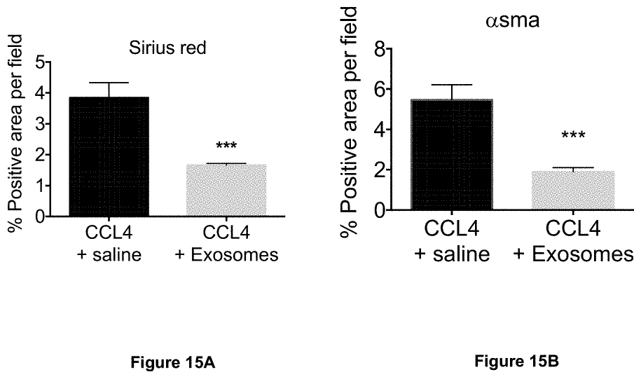

Liver fibrosis was induced in adult mice aged 8-12 weeks by 3× weekly intraperitoneal injection of carbon tetrachloride for 12 weeks. At week 8, exosomes (1 μg) were twice weekly injected. The results are shown in FIGS. 15A and B. Fibrobiotic cells were determined using the Sirius red assay and the α-smooth muscle action (SMA) expression assay. α-SMA plays a role in fibroblast contractility. α-SMA expression was determined using standard assays and Sirius red or α-SMA positive areas were measured per field. The inflammatory macrophage protein, CCL14, was used. CCL14+exosomes resulted in significant less fibrotic cells per field compared to CCL14+saline control (FIGS. 15A and B).

### Example 8

### Proteomic Cargo

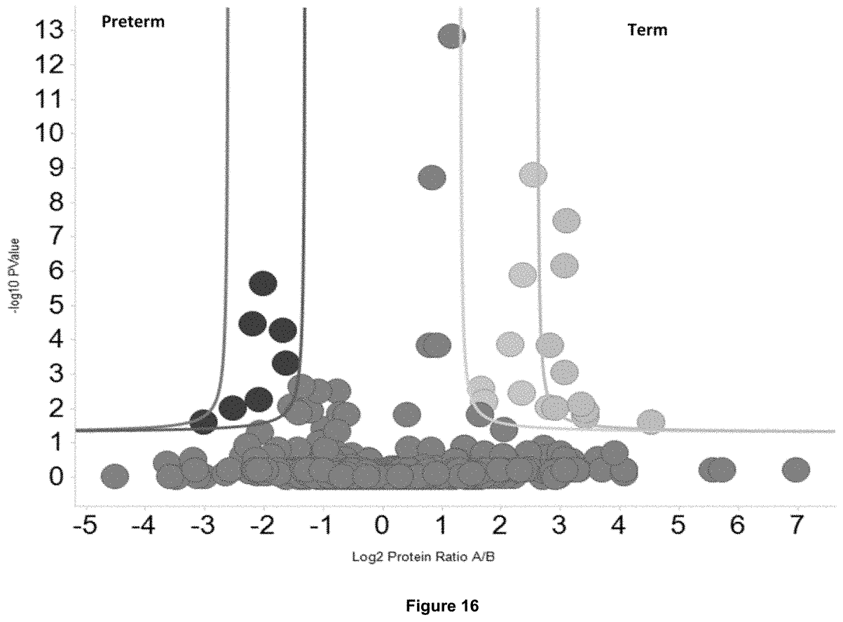

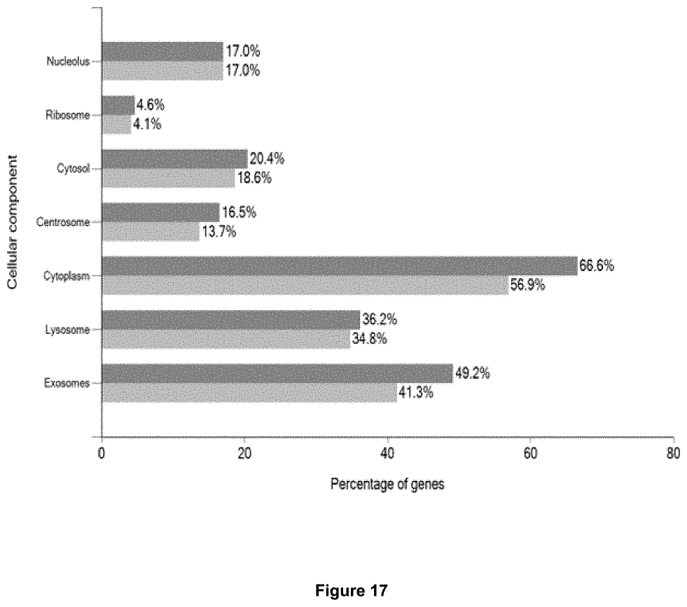

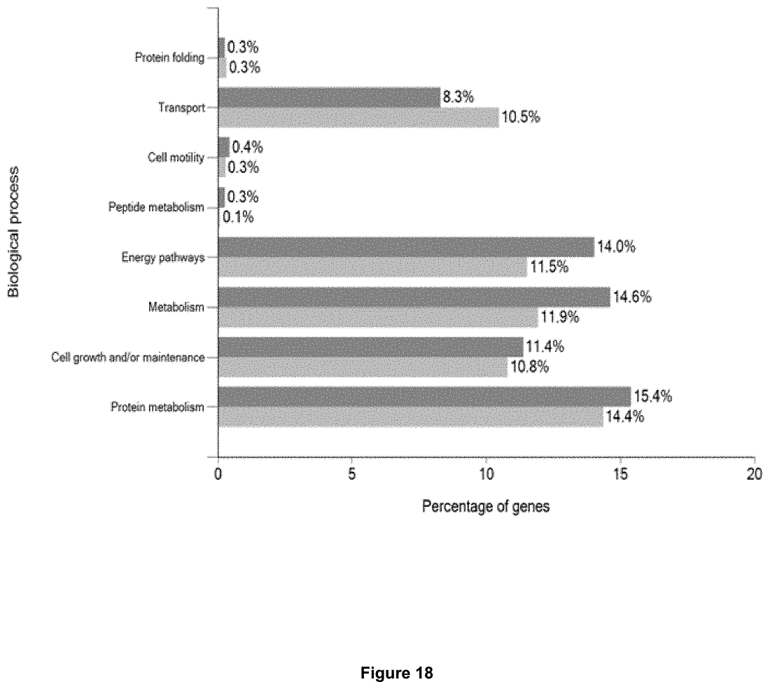

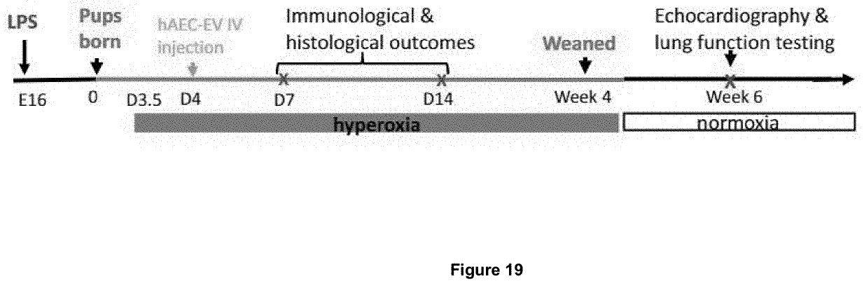

FIG. 16 shows that the proteomic cargo between term exosomes and preterm hAECs is about the same. There was more of a difference between the proteomic cargo of term versus preterm hAECs. Proteins tested are listed in Tables 2a and 2b. A useful cellular component comparison is shown in FIG. 17 between hAEC and total MSC. FIG. 18 also compares biological processes between hAECs and total MSC.

TABLE 2a
Proteomic cargo in amniotic exosomes: Proteins common to MSCs

| Search ID/Accessions | Gene Symbol | Entrez Gene ID | Gene Description |
|---|---|---|---|
| PGK2 | PGK2 | 5232 | phosphoglycerate kinase 2 |
| CAND2 | CAND2 | 23066 | cullin-associated and neddylation-dissociated |
| | | | 2 (putative) |
| CCDC80 | CCDC80 | 151887 | coiled-coil domain containing 80 |
| RAB12 | RAB12 | 201475 | RAB12, member RAS oncogene family |
| RAD23B | RAD23B | 5887 | RAD23 homolog B (S. cerevisiae) |
| DBN1 | DBN1 | 1627 | drebrin 1 |
| STRAP | STRAP | 11171 | serine/threonine kinase receptor associated |
| | | | protein |
| UBA6 | UBA6 | 55236 | ubiquitin-like modifier activating enzyme 6 |
| PEA15 | PEA15 | 8682 | phosphoprotein enriched in astrocytes 15 |
| GLOD4 | GLOD4 | 51031 | glyoxalase domain containing 4 |
| COPE | COPE | 11316 | coatomer protein complex, subunit epsilon |
| BCAT1 | BCAT1 | 586 | branched chain amino-acid transaminase 1, |
| | | | cytosolic |
| FSTL1 | FSTL1 | 11167 | follistatin-like 1 |
| DYNC1LI2 | DYNC1LI2 | 1783 | dynein, cytoplasmic 1, light intermediate |
| | | | chain 2 |
| GPS1 | GPS1 | 2873 | G protein pathway suppressor 1 |
| SPATA5 | SPATA5 | 166378 | spermatogenesis associated 5 |
| COPS5 | COPS5 | 10987 | COP9 signalosome subunit 5 |
| GYG1 | GYG1 | 2992 | glycogenin 1 |
| TCEB2 | TCEB2 | 6923 | transcription elongation factor B (SIII), |
| | | | polypeptide 2 (18 kDa, elongin B) |
| APOC2 | APOC2 | 344 | apolipoprotein C-II |
| MAPK3 | MAPK3 | 5595 | mitogen-activated protein kinase 3 |
| RUFY1 | RUFY1 | 80230 | RUN and FYVE domain containing 1 |
| HDLBP | HDLBP | 3069 | high density lipoprotein binding protein |
| TTC37 | TTC37 | 9652 | tetratricopeptide repeat domain 37 |
| UBA2 | UBA2 | 10054 | ubiquitin-like modifier activating enzyme 2 |
| NCS1 | NCS1 | 23413 | neuronal calcium sensor 1 |
| CAV2 | CAV2 | 858 | caveolin 2 |
| TM9SF4 | TM9SF4 | 9777 | transmembrane 9 superfamily protein |
| | | | member 4 |
| EIF5B | EIF5B | 9669 | eukaryotic translation initiation factor 5B |
| TXNL1 | TXNL1 | 9352 | thioredoxin-like 1 |

TABLE 2b
Proteomic cargo in amniotic exosomes: Proteins unique to hACEs

| Search ID/Accessions | Gene Symbol | Entrez Gene ID | Gene Description |
|---|---|---|---|
| TNXA | | | |
| TES | TES | 26136 | testis derived transcript (3 LIM domains) |
| NPEPPSL1 | | | |
| UPK3BL | | | |
| POLR2J3 | | | |
| DKFZp586I031 | | | |
| SEPT14 | SEPT14 | 346288 | septin 14 |
| DKFZp313C1541 | | | |
| SLC2A14 | SLC2A14 | 144195 | solute carrier family 2 (facilitated |
| | | | glucose transporter), member 14 |
| PPIAL4C | | | |
| PPIAL4E | | | |
| PPIAL4D | PPIAL4D | 645142 | peptidylprolyl isomerase A (cyclophilin |
| | | | A)-like 4D |
| CRABP1 | CRABP1 | 1381 | cellular retinoic acid binding protein 1 |
| Sep-02 | | | |
| TPPP3 | TPPP3 | 51673 | tubulin polymerization-promoting |
| | | | protein family member 3 |
| Sep-08 | | | |
| ARPC4-TTLL3 | | | |
| KIF5C | KIF5C | 3800 | kinesin family member 5C |
| KIF5A | KIF5A | 3798 | kinesin family member 5A |
| NSFL1C | NSFL1C | 55968 | NSFL1 (p97) cofactor (p47) |
| PERP | PERP | 64065 | PERP, TP53 apoptosis effector |
| SKP1 | SKP1 | 6500 | S-phase kinase-associated protein 1 |
| ALPPL2 | ALPPL2 | 251 | alkaline phosphatase, placental-like 2 |
| ALPI | ALPI | 248 | alkaline phosphatase, intestinal |
| PCYT2 | PCYT2 | 5833 | phosphate cytidylyltransferase 2, |
| | | | ethanolamine |
| CDH3 | CDH3 | 1001 | cadherin 3, type 1, P-cadherin (placental) |

### Example 9

### Exosomes Promote Myelination

Amniotic exosomes are tested in animal models of multiple sclerosis. It is expected that the exosomes will promote remyelination and be useful in the treatment of multiple sclerosis as well as other conditions such as optic neuritis, Devic's disease, transverse myelitis, acute disseminated encephalomyelitis and adrenoleukodystrophy and adrenomyeloneuropathy.

### Example 10

### Exosome Activity

Exosome isolated from the conditioned media of human amnion epithelial cells have immunomodulatory and pro-regenerative effects. The amniotic exosomes contain (amongst) other factors), high level of HLA-G.

Immunosuppressive effectives of amniotic exosomes correspond to the gestational age of the donor. This corresponds to donor potency associated with gestational age, which we have previously published in (Lim et al. (2013) supra).

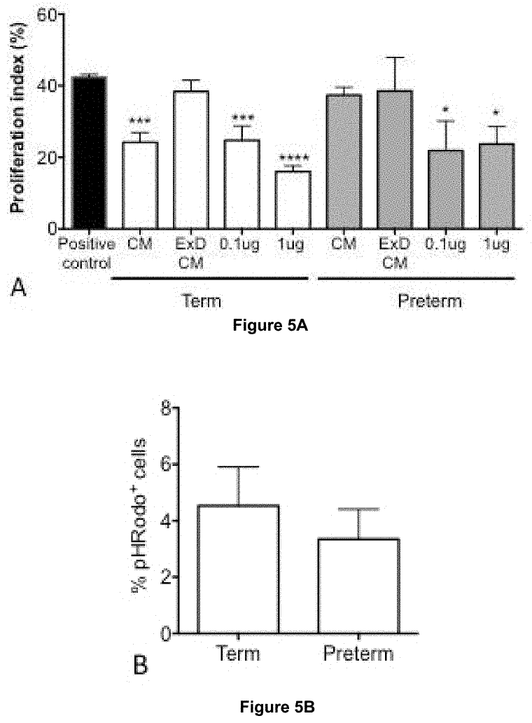

Amniotic exosomes reverse lung injury in a neonatal mouse model of bronchopulmonary dysplasia. Intravenously injected exosomes significantly improve tissue:airspace ratio compared to saline, and consistent to our in vitro, term amniotic exosomes were superior to preterm exosomes in their ability to mitigate BPD related lung damage. This is associated with an activation of the endogenous stem cell niche of the lungs i.e. bronchioalveolar duct junction. The results are shown in FIG. 5. It is proposed that the amniotic exosomes will be useful in the treatment of lung fibrosis and fibrosis of other organs.

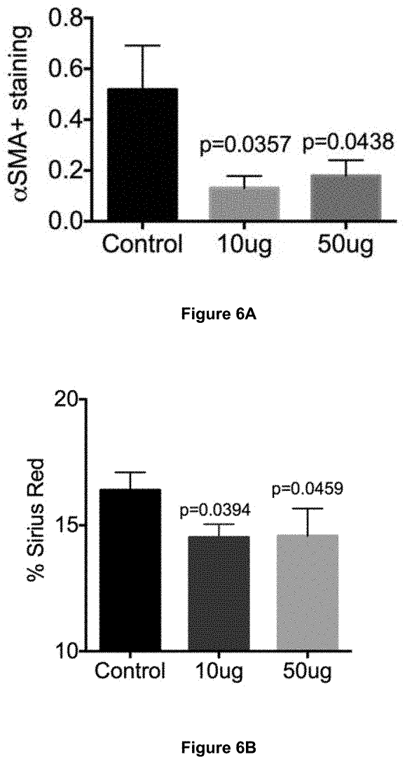

Amniotic exosomes reverse established lung inflammation and fibrosis in a mouse model of bleomycin induced lung fibrosis. Intranasal administration of amniotic exosomes 7 days post bleomycin challenged significantly reduced the percentage of activated myofibroblasts (α-smooth muscle actin positive) in the lungs. This was consistent with the reduction in collagen deposition in the lungs. The results are shown in FIG. 6.

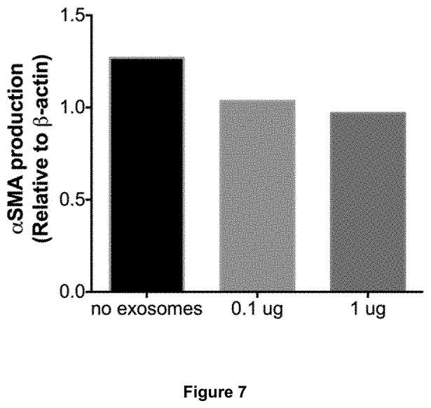

Amniotic exosomes directly reverse activation of primary human lung fibroblasts in vitro. When cultured in the presence of 5 ng/mL transforming growth factor β, amniotic exosomes decreased protein levels of α-smooth muscle actin within 24 hours. The results are shown in FIG. 7.

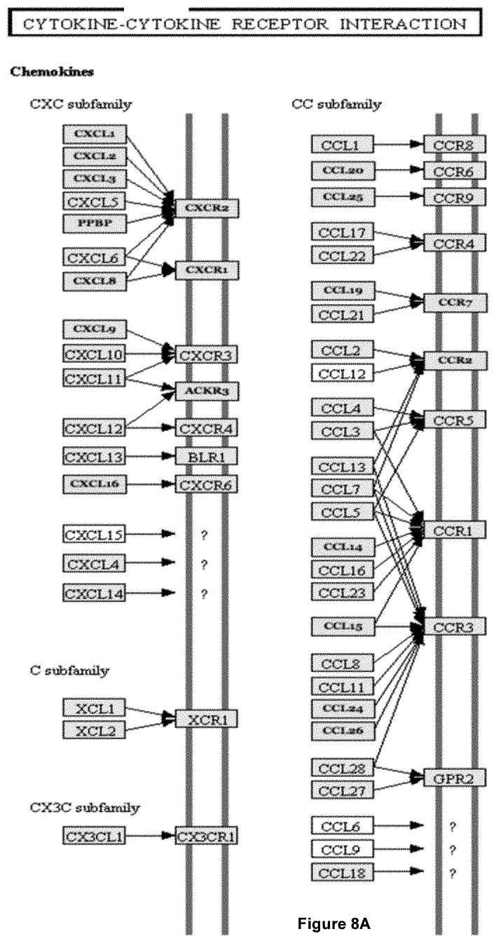

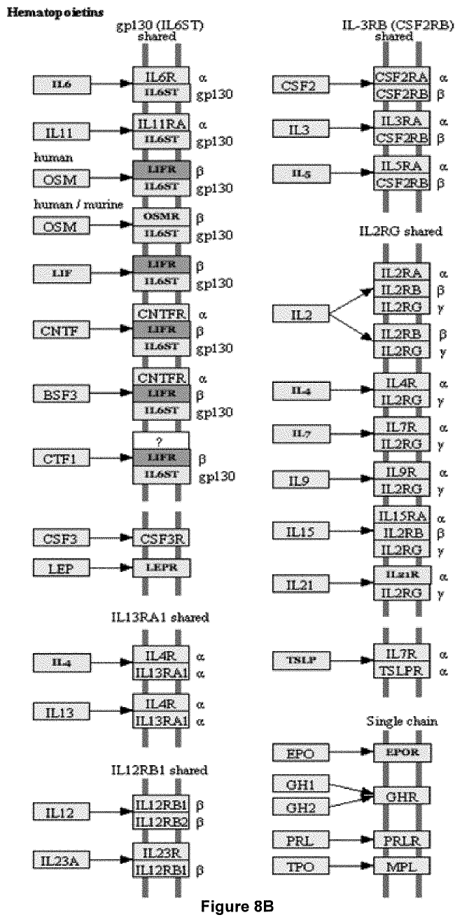

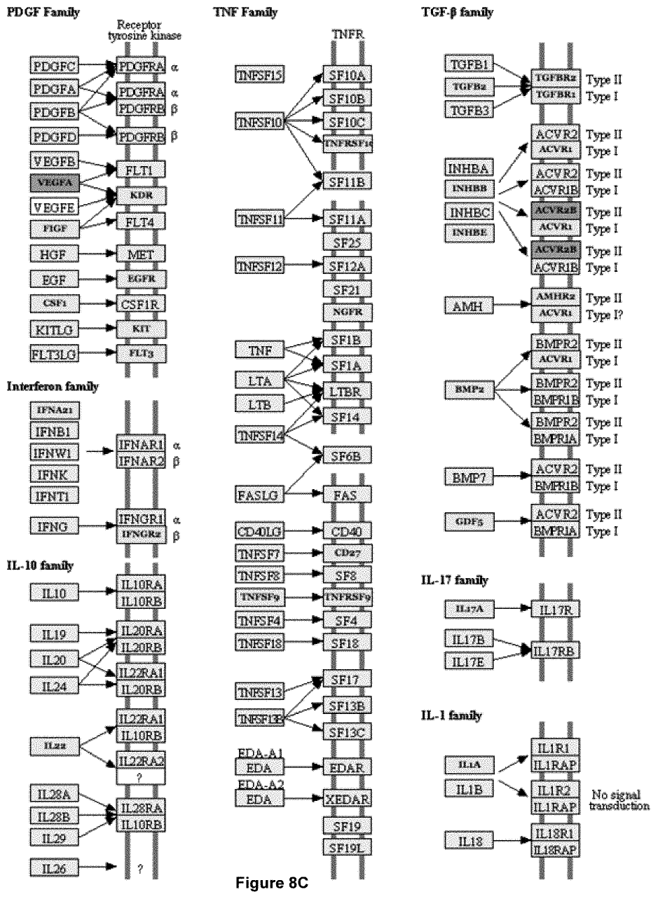

Amniotic exosomes contain miRNAs that target the cytokine-cytokine receptor signaling pathways as shown in FIG. 8, where yellow boxes indicate a target by one or more miRNAs.

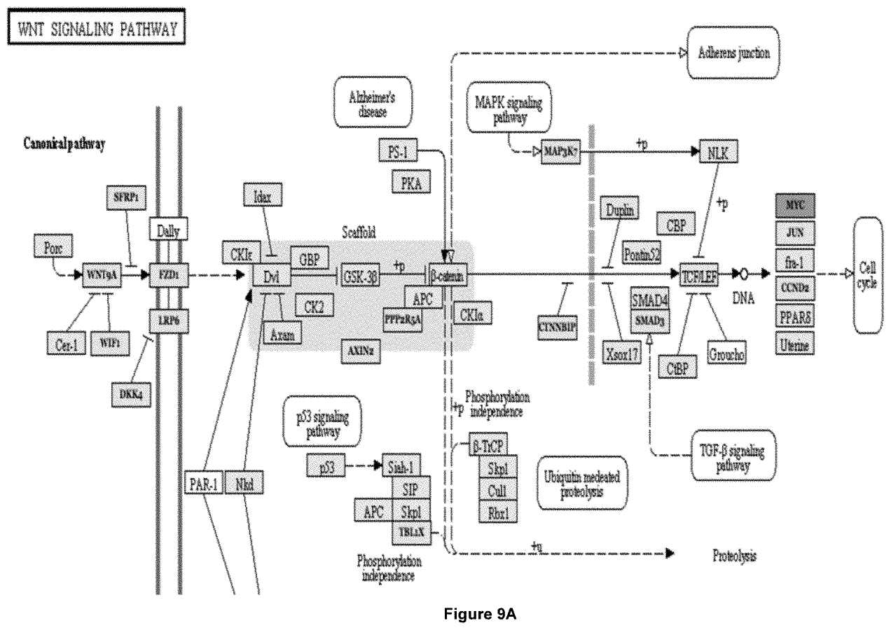

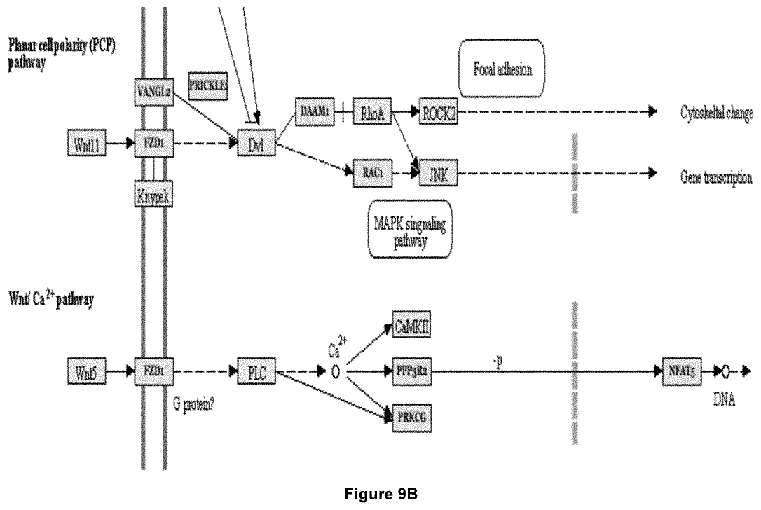

Amniotic exosomes contain miRNAs that target the Wnt signaling pathways as shown in FIG. 9, where yellow boxes indicate a target by one or more miRNAs.

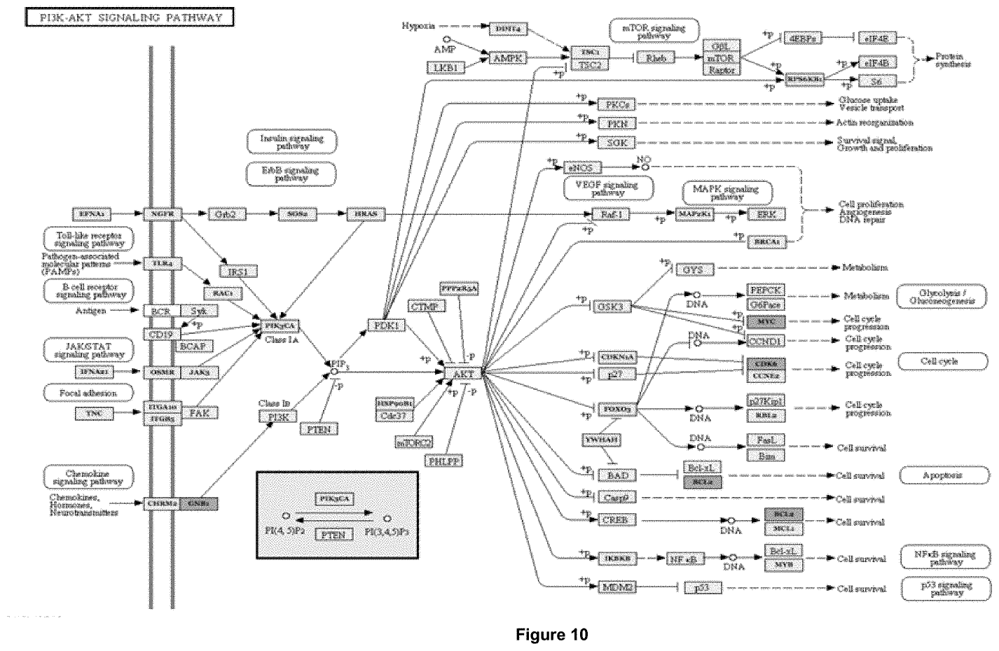

Amniotic exosomes contain miRNAs that target the PI3K-Akt signaling pathways as shown in FIG. 10 where yellow boxes indicate a target by one or more miRNAs.

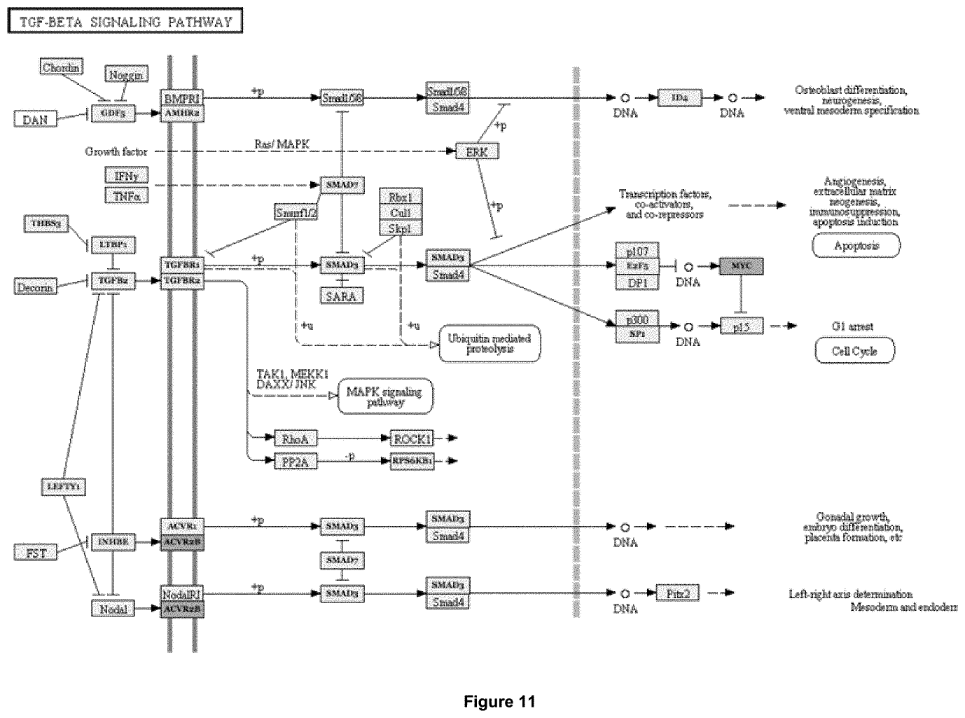

Amniotic exosomes contain miRNAs that target the TGFβ signaling pathways as shown in FIG. 11 where yellow boxes indicate a target by one or more miRNAs.

It is clear that amniotic exosomes are as, if not more, effective than AECs such as hAECs and have a great capacity to induce cellular and molecular repair mechanisms in a diverse range of physiological and neural processes.

### Example 11

### Characterisation and Potency of Term and Preterm hAEC-EVs

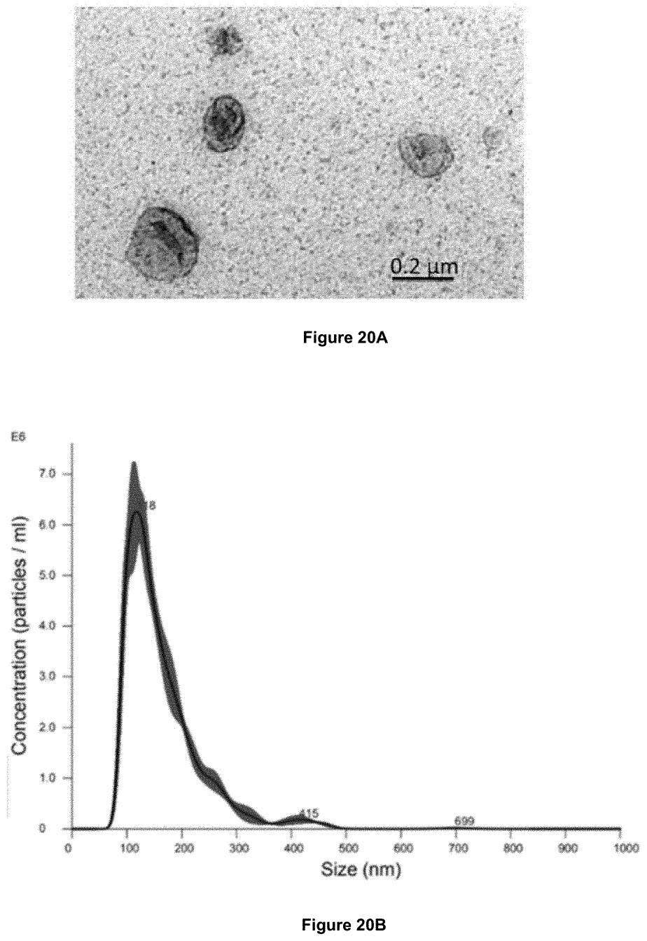

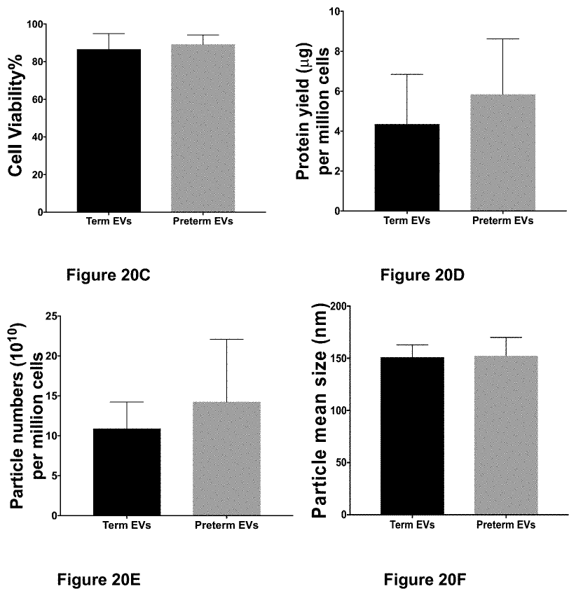

The terms “exosome” and “extracellular vesicles” (EV) are used interchangeably herein. hAEC-EVs typically exhibited a cup shaped morphology across all cell lines regardless of gestational age of the donor (FIGS. 20A-B). No significant differences in the cell viability, EV protein yield, EV particle number, particle mean size and size distribution between term and preterm hAECs was observed (FIGS. 20C-F).

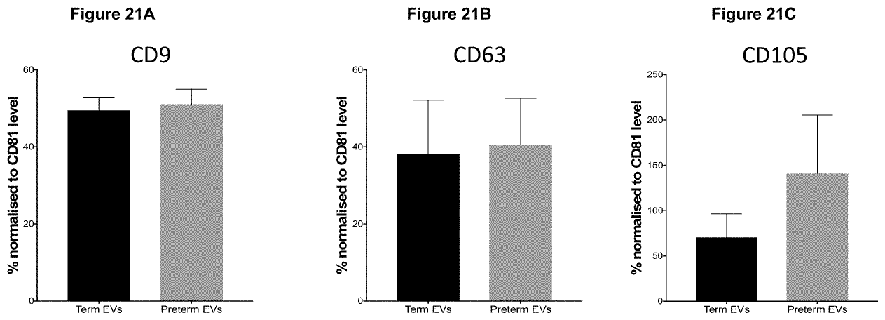

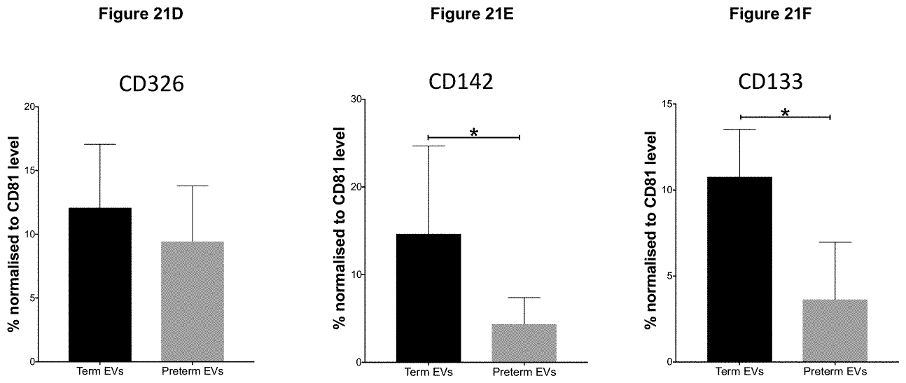

MACSPlex results were normalized to the fluorescence intensity of CD81. It showed that hAEC-EVs present EV common tetraspanins CD9 CD63, CD81, as well as soluble VEGF receptor CD105, and epithelial cell marker CD326. There were no differences in expression levels of these surface epitopes between hAEC-EVs from term and preterm donors (FIG. 21A-D). However, hAEC-EVs from term donors expressed higher levels of CD142 and CD133 compared to preterm EVs (FIG. 21E-F).The T cell proliferation index is the average number of divisions of the T cells. It showed that preterm EVs suppressed T cell proliferation more significantly than term EVs with lower proliferation index (FIG. 22A). However, term EVs improved macrophage phagocytosis more significantly than preterm EVs (FIG. 22B).

### Example 12

### Short-Term Outcomes of BPD Experiment

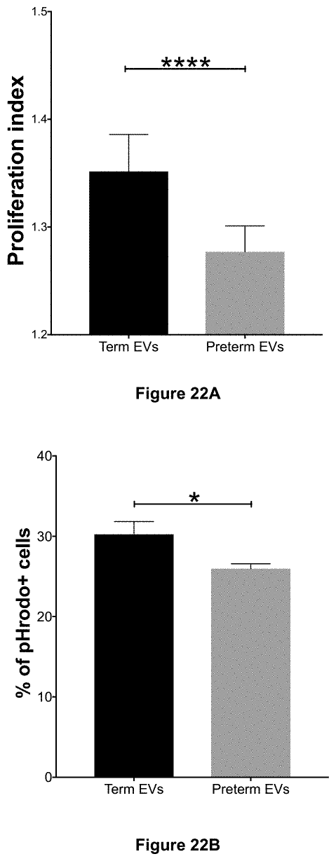

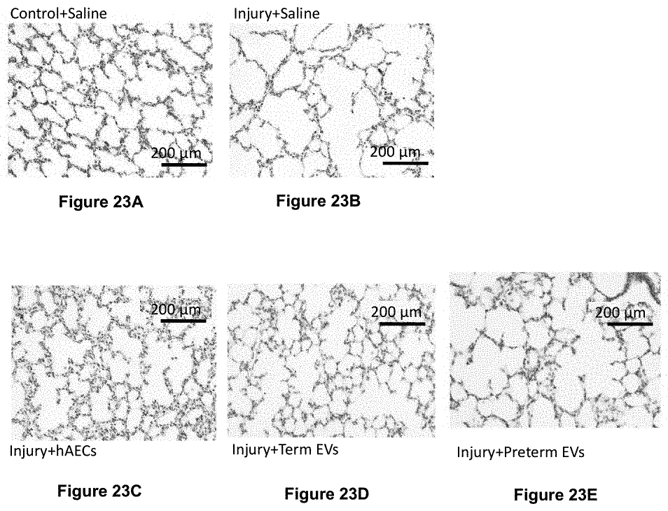

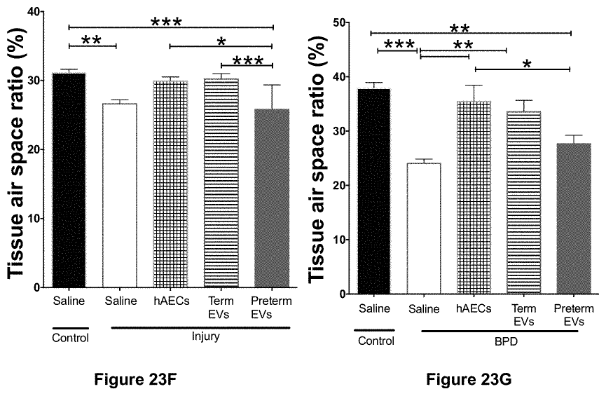

hAEC-EVs from Term Donors Ameliorated Alveolar Simplification: The combination of intra-amniotic LPS and neonatal hyperoxia significantly reduced tissue-to-air space ratio by PND 7 and 14 (p<0.001 and p<0.01) compared to control groups (FIG. 23A-C). Only hAEC-EVs from term donors improved tissue-to-air space ratio such that they were not significantly different to the injured group and hAEC treatment group by PND7 (FIG. 23B). Furthermore, hAEC-EVs from term donors mitigated the injury by PND14 as seen by the improvement in tissue-to-air space ratio, such that they were comparable to the control group (FIG. 23A, 23C). In contrast, tissue-to-air space ratio of animals given hAEC-EVs from preterm donors remained significantly lower than healthy controls (p<0.001, FIG. 23A, 23C). The tissue to airspace ratio achieved by the hAEC was higher than both EV treatment groups by PND7 (p<0.05). The outcomes achieved by either hAECs or term EVs was comparable by PND14 (FIG. 23B-C).

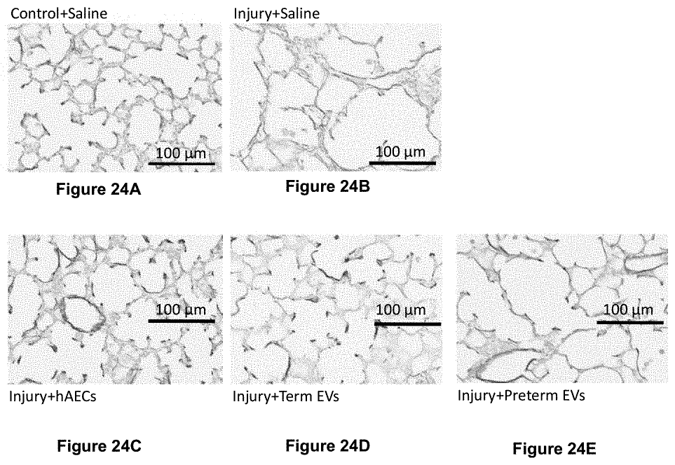

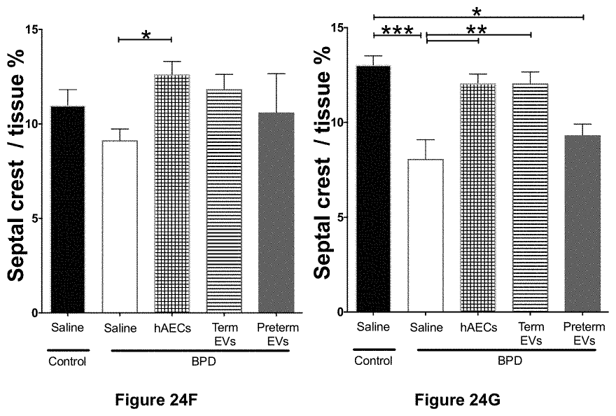

Term hAEC-EV Treatment Improved Secondary Septal Crest Density: By PND7, term hAEC treatment improved secondary septal crest density such that they were comparable to control (FIG. 24B). By PND14, secondary septal crest density was significant decreased in the injury group (p<0.001, FIG. 24A, 7C) and this was only mitigated in the animals that received term but not preterm EVs (FIG. 24C).

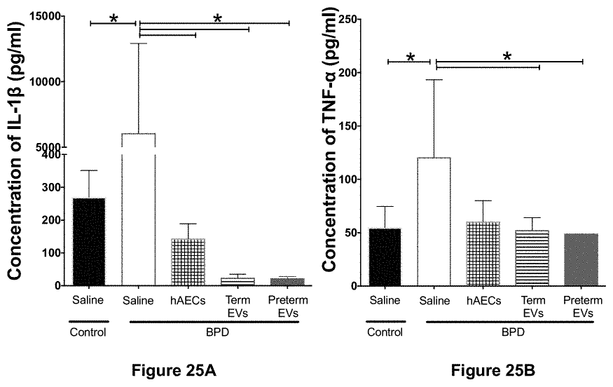

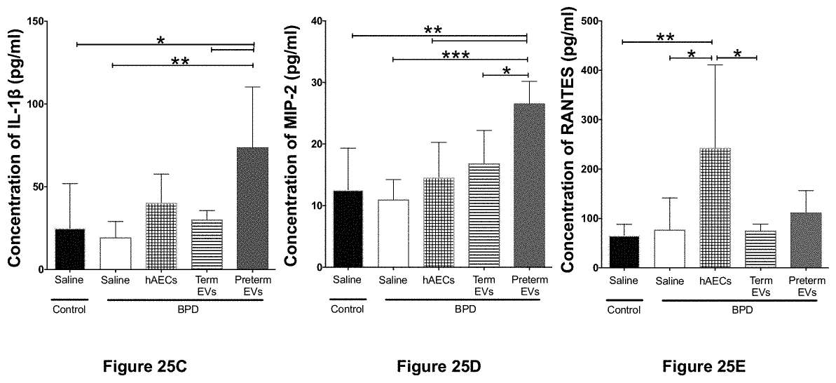

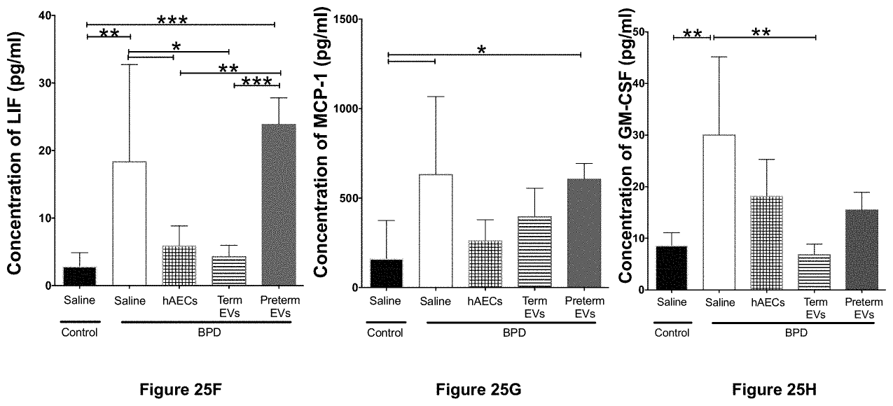

Term hAEC-EV Treatment Decreased Lung Inflammation: By PND7, both term and preterm EV administration reduced IL-1β and TNF-α to control levels (p<0.05, FIG. 25A-B), which is comparable to hAEC treatment. Interestingly, the preterm EV group had higher IL-1β and MIP-2 levels compared to other groups by PND14 (FIG. 25C-D). The level of RANTES was elevated in hAEC treatment group, but not in the term EV group (p<0.05, p<0.01, FIG. 25E). The levels of LIF, MCP-1 and GM-CSF in the injury group were higher than control, and both hAECs and term EVs reduced them to control levels, but the levels of LIF and MCP-1 remained high in preterm EV treated mice (p<0.05, p<0.01, p<0.001, FIG. 25F-H). Levels of TNF-α and IL-6 were below the limit of detection.

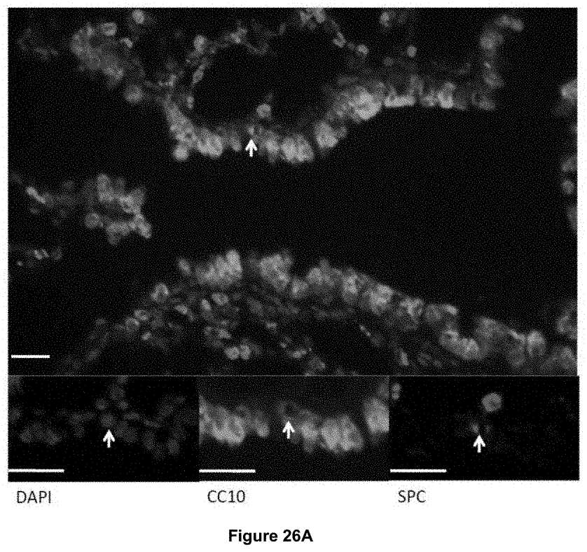

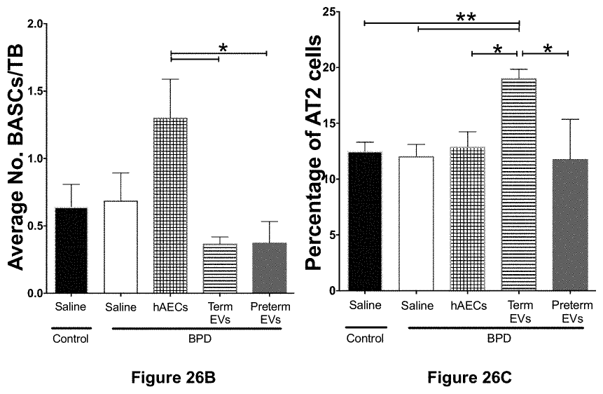

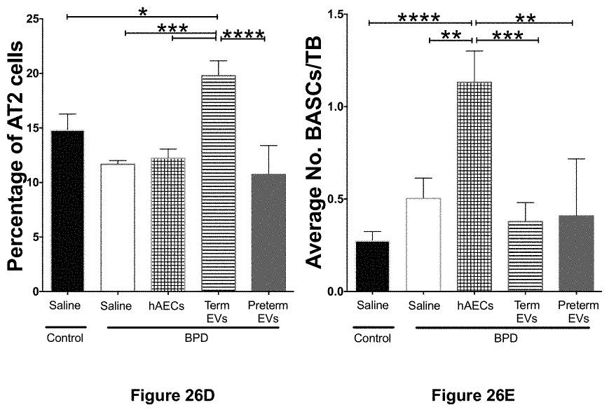

Term hAEC-EV Treatment Induced Type II Alveolar Cell but not Bronchoalveolar Stem Cell Proliferation: BASCs stain double positive for pro-SPC/CC10 and are located at the terminal bronchioles. AT2s stained positive for pro-SPC and are located throughout the parenchyma (FIG. 26A). Hyperoxia-LPS induced lung injury had no effect on BASC and AT2 cell activation. The average number of BASCs per terminal bronchiole did not change in response to either EV group on both PND7 and 14 (FIG. 26B-C), which was in contrast to hAEC treatment. However, the percentage of AT2 cells was significantly increased in the term EV treatment group at both time points compared to control group (p<0.05 and p<0.01). In contrast, there was no significant change in either preterm EV or hAEC treatment groups (FIG. 26D-E).

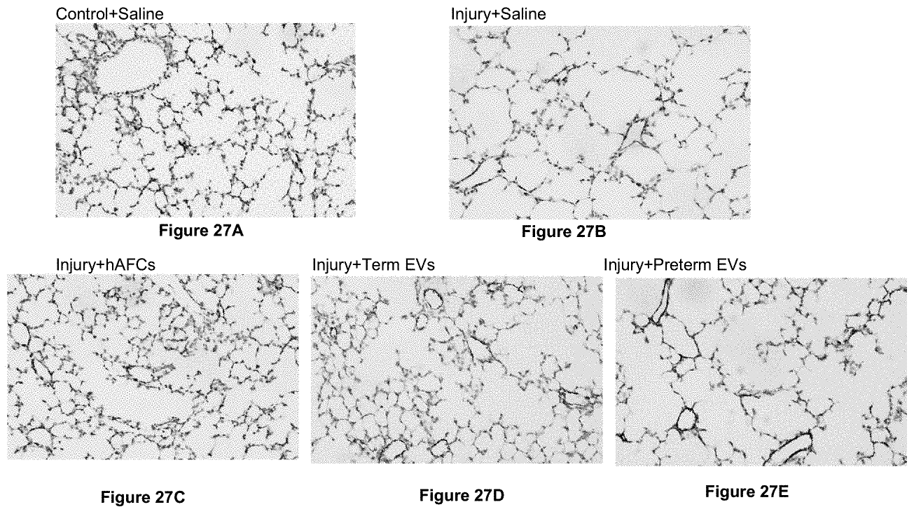

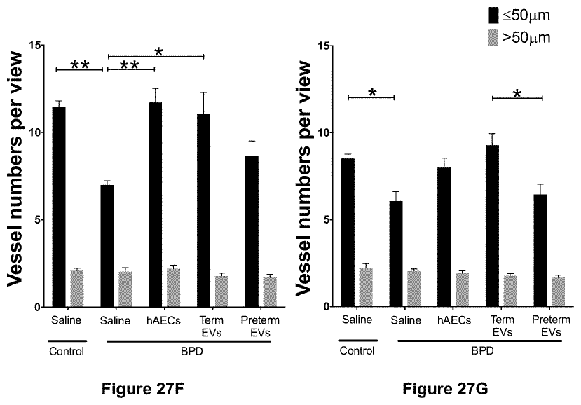

Term hAEC-EV Treatment Increased the number of vWF positive pulmonary vessels: Here we observed that the numbers of small pulmonary vessels (<50 μm in diameter) were significantly decreased on both PND7 and 14 (p<0.01 and p<0.05, FIG. 27A-C). hAEC and term EV treatment restored pulmonary vascularisation to control levels, but this was not achieved by preterm EV treatment. (FIG. 27B-C). Larger blood vessels (>50 μm) were unaffected across all experimental groups.

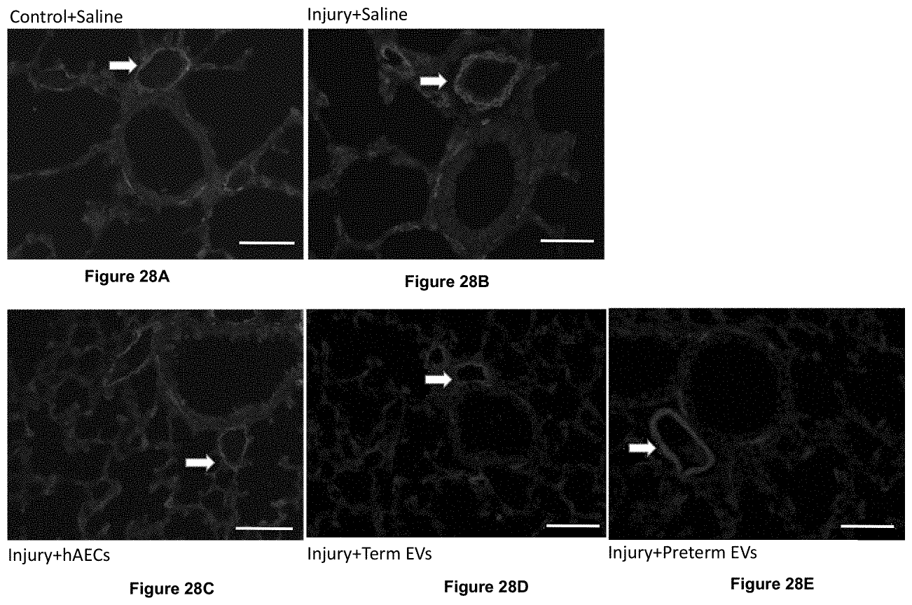

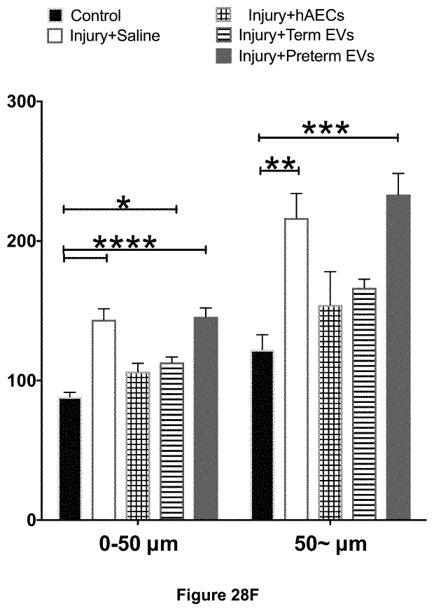

Term hAEC-EV Treatment Reduced Peripheral Pulmonary Arterial Remodeling: The arterial medial layer was stained with α-SMA (FIG. 28A). The thickness of the arterial medial layer was significantly increased by PND14, but this was attenuated by in the hAEC and term EV treatment groups (p<0.01, p<0.001, p<0.0001, FIG. 28B).

### Example 13

### Short-Term Outcomes of BPD Experiment

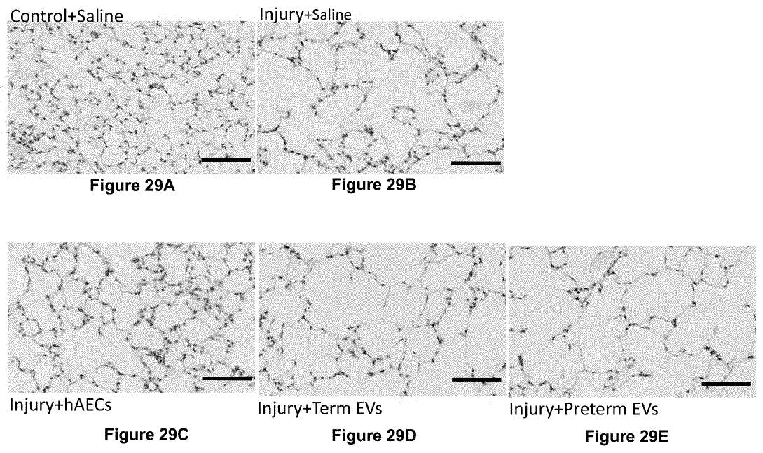

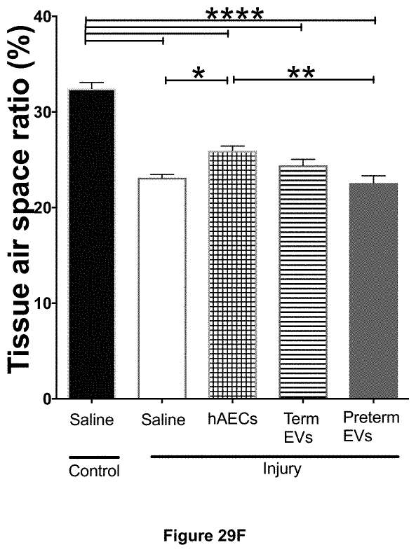

Term hAEC-EV Treatment Improved Lung Tissue-To-Air Space Ratio: Reduced tissue-to-airspace ratio persisted until week 6 but was significantly improved by hAEC treatment. It was also improved by term EV (FIG. 29A-B).

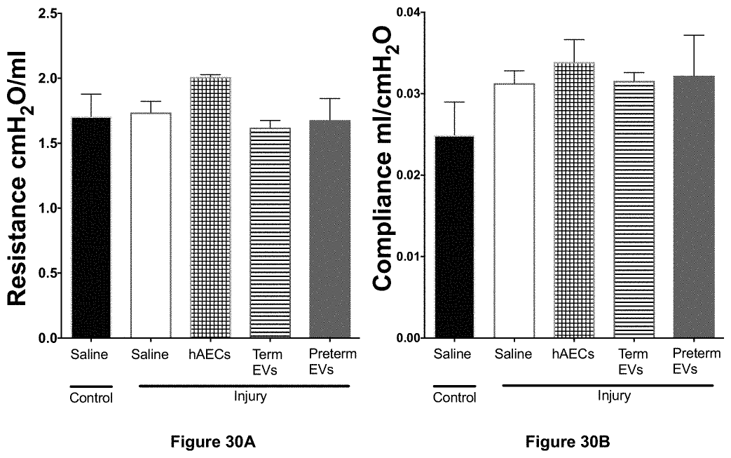

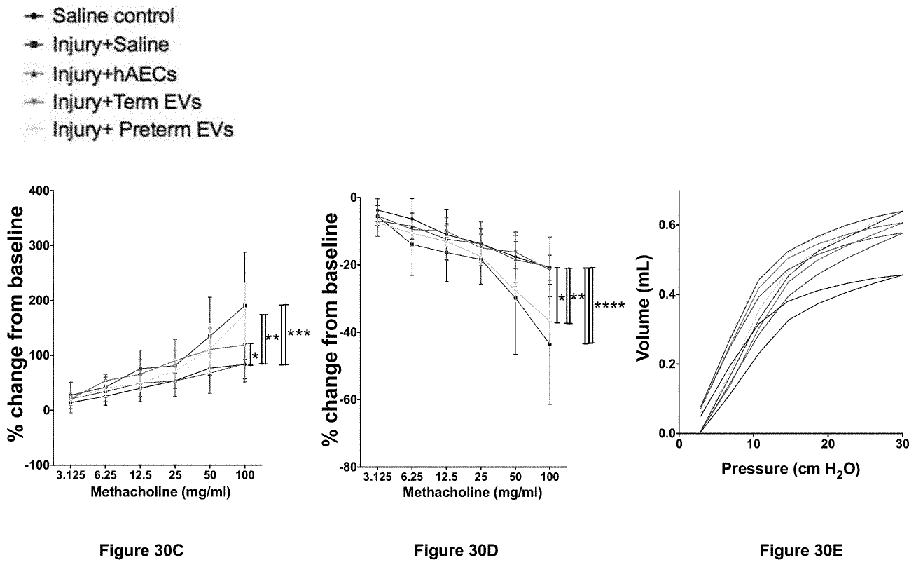

Term hAEC-EV Treatment Prevented the Increase of Airway Responsiveness: Lung function test showed that baseline resistance (Rrs) and compliance (Crs) did not significantly change between groups (FIG. 30A-B). When challenged with 100 mg/mL methacholine, the injured mice showed significantly increased Rrs and decreased Crs compared to the control (p<0.001 and p<0.0001 respectively, FIG. 30C-D). hAEC treatment restored Rrs and Crs to control levels. Term EV treatment increased Crs to healthy mice level, and decreased Rrs to the level that was between control and injured groups (p<0.05, FIG. 30C-D). The pressure-volume (PV) loop is generated by changes to lung volume during a respiratory cycle. Compared to control, the PV loop of the injured group saw a significant upward shift, indicating increased lung compliance and suggestive of reduced tissue elasticity. Both hAEC and term EV treatment returned the loop downwards such that the PV loop sat between the control and injured groups. The PV loop position of mice that received preterm EVs remained higher than control. (FIG. 30E).

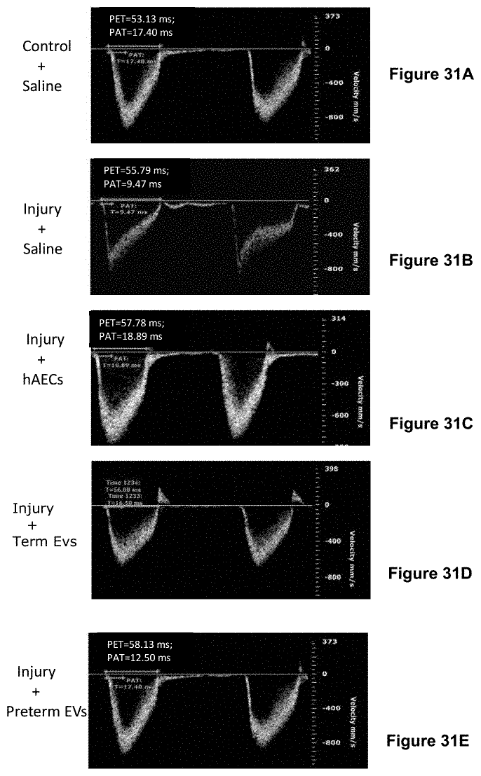

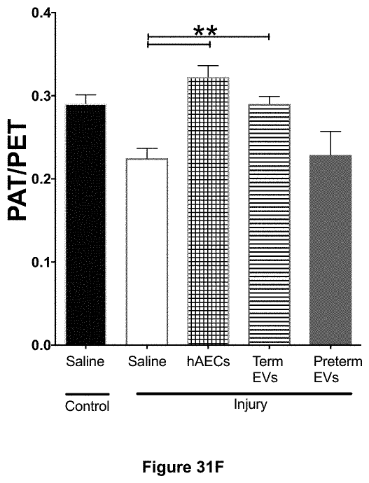

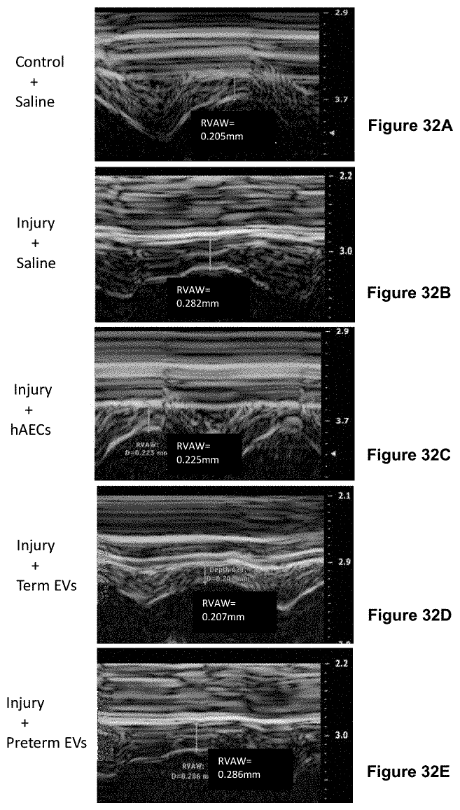

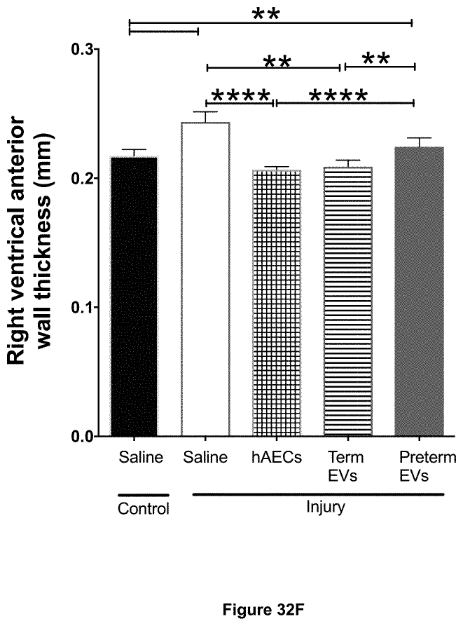

Term hAEC-EV Treatment Prevented Pulmonary Hypertension and Right Ventricular Hypertrophy: Echocardiography showed that the injury group developed pulmonary hypertension with reduced pulmonary artery acceleration to ejection time (PAT/PET, FIG. 31A-B), and right ventricle hypertrophy with thickened RVAW (FIG. 32A-B) by week 6. These were attenuated by only with hAEC treatment or term EV treatment (FIG. 31A-B, 32A-B).

### Example 14

### Delayed Post-Stroke Administration of Human Amnion Epithelial Cells-Derived Exosomes Improves Outcomes

Mouse model of stroke: A total of 79 C57B16/J male mice aged 8-10 weeks and weighing 21-30 g used for this study. Mice were housed individually post-surgery and had access to standard chow pellets and water. Mice were excluded if they died either during or after stroke surgery (before 24 hour post-stroke testing), or if they lacked sufficient functional impairments 24 hour post-stroke.

Photothrombotic stroke: Focal ischaemia was induced in the right primary motor cortex for 15 min using the photothrombotic model of stroke as described previously (Evans et al., 2018; Neural Regen Res. 2018 August; 13(8): 1346-1349). Upon completion, mice were allowed to regain consciousness before being returned to their cages. Mice undergoing sham surgery were subjected to the same procedure, except they were not injected with Rose Bengal.

Administration of hAEC-derived exosomes: hAEC-derived exosomes were prepared as described above. Mice undergoing photothrombotic stroke were randomly administered with one of the following treatments via tail vein injection: saline (vehicle) or 10 μg of hAEC-derived exosomes; this dose was chosen as our group has recently shown that i.v. administration of 10 μg of hAEC-derived exosomes is neuroprotective following stroke. Treatments were administered at 24 h post-stroke, with an additional injection of respective treatment at 14 day post-stroke for the 28 day study.

Functional testing: Functional tests assessing sensorimotor deficits included the hanging wire, cylinder and adhesive removal tests, which were conducted on day 0 (prior to surgery), and days 1, 3 and 7 post-surgery for the 7 d study, and continued with assessments on days 14, 21 and 28 for the 28 d study. Body weights were recorded on the same days as functional testing.

Hanging wire test: This test was used to assess the forelimb grip strength and coordination, which are controlled by the M1 motor cortex region (Balkaya et al., 2013; J. Cereb Blood Flow Metab. 2013 March; 33(3): 330-338). Mice were suspended by their forelimbs on a thin metal wire stretched between two posts 60 cm above a soft landing (bubblewrap). The time until mice fell off the wire (latency to fall) was measured, with a maximum time of 300 s (indicating the animal did not fall). The average time of 3 trials with a 5 min rest in between was calculated for each testing time point. Mice were excluded from the study if their average time 24 post-stroke was greater than 120 s.

Cylinder test: In addition, the cylinder test was used to assess motor function in terms of forelimb symmetry and use during vertical exploration and landing (Balkaya et al., 2013). Mice were placed in a glass cylinder, and their activity was video recorded for 5 min. Four broad outcomes were scored: 1) the first forelimb to touch the wall during vertical exploration/rearing (initial touch), 2) all other forelimb touches on the wall during rearing (all subsequent touches), and 3) first forelimb to touch the floor during landing (landing). For each of these outcomes, the number of left, right or both paw touches were scored. To observe any affinity for one paw over the other, the percentage of left forepaw touches for the first three outcomes was calculated using the following formula:

$$\% of lef t forepaw touches = ( \frac{\# left touches + \# both touches}{\# left touches + \# right touches + ( \# both touches \times 2 )} ) \times 10$$

The left forepaw usage was specifically analysed as this was considered the “impaired paw” given that a stroke was induced in the right M1 region. Lastly, 4) the number and length of time (s) the left or right paw was “clenched” was recorded. Clenching was considered the act of holding one forepaw close towards the body or chest while rearing or in contact the ground.

Adhesive Removal Test: This test assessed mainly sensory function (Balkaya et al., 2013; J. Cereb Blood Flow Metab. 2013 March; 33(3): 330-338). A small circular sticker was applied to each forepaw, after which the mice were placed inside a transparent Perspex box. The time until both stickers were removed was recorded and averaged from 3 trials, with a 5 min rest period in between each trial.

Histological analyses: Mice were euthanised via isoflurane inhalation followed by decapitation after testing on the 7^{th} or 28^{th} day post-surgery. Brains were removed and slow-frozen in liquid nitrogen, followed by storage in −80° C. for histological assessment. Serial coronal brain sections spanning the infarct (typically within following range: bregma +2.34 mm to +0.02 mm, separated by 240 μm) were collected for infarct volume analysis and immunofluorescence using a cryostat (Leica Biosystems) and were thaw-mounted onto poly-L-lysine coated (1 mL 0.1% poly-L-lysine in 50 mL dH₂O) glass slides.

Infarct volume analysis: Infarct volumes were analysed by staining 30 μm sections in 0.1% thionin (Sigma, Australia; diluted in acetic acid; Ajax Chemicals, Australia) for 2 min, followed by quick rinsing in dH₂O, and consecutive 2 min rinses in 70% and 100% ethanol. Slides were then dried and cover-slipped with xylene (Merck, Australia) and Distyrene, Plasticiser and Xylene (DPX; Merck, Australia). After overnight drying, photos of each section were taken using a charge coupled device (CCD) camera (Cohu, Inc., San Diego, Calif., USA) mounted above a light box (Biotec-Fischer Colour Control 5000, Reishkirchin, Germany) and the IS Capture imaging software. Images were then analysed using the ImageJ software (NIH, Bethsada, Md., USA). Infarct volume was then calculated as previously described in Brait et al. (2010; J Cereb Blood Flow Metab.; 30(7):1306-17).

Immunofluorescence analyses: Immunofluorescence was conducted on 10 μm sections representing different regions of the brain. Frozen sections were air-dried for 5-10 min, fixed in either acetone or 4% paraformaldehyde (Merck, Australia) for 15 min and then washed in 0.01M PBS (3×10 min). A wax pen (Agilent Dako, CA, USA) was used to draw around the sections. Sections were then blocked with 10% normal goat serum (Abcam; Cambridge, USA, diluted in 0.01 M PBS) or 5% bovine serum albumin (BSA; Sigma) in 0.02% Tween-20 (Sigma) for 60 min to prevent non-specific binding. Sections were incubated with the following primary antibodies diluted with either antibody diluent or 1% BSA in 0.02% Tween-20; cleaved caspase-3 (1:200, Abcam, Cambridge, USA, myeloperoxidase (MPO; 1:200, Abcam, Cambridge, USA), CD3 (1:200, Abcam, Cambridge, USA), B220 (1:100, Invitrogen), CD206 (1:200, Abcam, Cambridge, USA), F4/80 (1:100, BioRad), Iba-1 (1:400, Abcam, Cambridge, USA), Ki-67 (1:500, NeoMarkers, Thermo Scientific), von Willebrand factor (vWF; 1:750, Abcam, Cambridge, USA), glial fibrillary acidic protein (GFAP; 1:400, Sigma) or doublecortin (DCX; 1:500, Abcam, Cambridge, USA) overnight at either room temperature or at 4° C. The following day, sections were washed in 0.01M PBS (3×10 min) and incubated with the appropriate secondary antibody (1:500, diluted with either antibody diluent or 0.05% Tween-20) from the following; goat anti-rabbit Alexa Fluor 594 or 488 IgGs, goat anti-rat Alexa Fluor 594 or 488 IgGs, or donkey anti-goat Alexa Fluor 488 IgG for 2-4 h at room temperature. Following incubation, sections were washed in 0.01M PBS (3×10 min) and dried with a kimwipe. Finally, 1-2 drops of VECTASHIELD mounting medium (Vector Laboratories, Burlingame, Calif.) were added onto the sections and slides were cover-slipped and sealed with nail polish. When mouse-derived primary antibodies were used against neuronal nuclei (NeuN; 1:500, Merck Millipore) and GAP-43 (1:400, Sigma), the Mouse-on-Mouse kit (Vector Laboratories) was used as per supplied protocol. Sections were imaged using an Olympus fluorescent microscope (BX51, Hamberg, Germany) and analysed using the ImageJ software (NIH). Cleaved caspase-3 and MPO were examined by counting the immunoreactive cells within the infarct. CD3 and B220 were examined by counting the immunoreactive cells both within and outside of the infarct. F4/80 and CD206 were examined by counting the number of co-localised cells both within and outside of the infarct, with the latter counts being expressed as a percentage of total F4/80-positive cells. Iba-1 was examined by counting the number of positive cells within three 200× fields of view (FOVs) per section. GAP-43 was examined by counting the number of immunoreactive growth cones within the infarct. Ki67 and NeuN were examined by counting the number of co-localised cells and expressed as a percentage of total Ki67-positive cells within six-eight 400× FOVs captured throughout the ischaemic hemisphere. vWF and GFAP were examined by counting the number of immunoreactive microvessels and astrocytes, respectively, in three 400× FOVs within the peri-infarct region. Finally, DCX was examined by measuring both the immunoreactive density within three 400× FOVs within the peri-infarct region, as well as the total immunoreactive area. Counts and measurements for all markers were averaged over six sections per brain.

Flow cytometry: After testing on the 7^{th} day, mice were euthanised via carbon dioxide inhalation and perfused with 0.01 M RNAse-free PBS solution with 0.2% clexane (400 IU, Sanofi Aventis) through the left ventricle. Blood was collected from the inferior vena cava, and the brain and spleen were removed, with the brain dissected and the right (ischaemic) hemisphere collected for analysis. These tissue were processed to obtain single-cell suspensions ready for staining as described previously (Chu et al., 2014; Journal of Cerebral Blood Flow & Metabolism, 34, 450-459). The final samples were incubated in a fluorophore-conjugated antibody mixture containing: CD45 (APC-Cy7, BioLegend, USA), CD11b (PacB, eBioscience, USA), CD3 (V500, eBioscience, USA), CD4 (FITC, BioLegend, USA), CD8 (BV785, BioLegend, USA), F4/80 (APC BioLegend, USA), CD206 (PE, BioLegend, USA) and CD19 (PacB, Invitrogen, USA) diluted in 0.01M PBS containing 0.5% BSA for 20 min in the dark. Additionally, single-colour compensation controls were prepared from UltraComp eBeads® (eBiosciences, USA), as well as fluorescence-minus-one controls for the F4/80 and CD206 antibodies, and an unstained tissue control from a spleen sample. Prior to machine analysis, the following were added to the tissue samples; 25 μL of CountBright counting beads (Invitrogen, USA) for normalisation and counting of absolute cell numbers, and 7-aminoactinomycin D (7AAD) for distinguishing live/dead cells. Samples were run on the BD LSR Fortessa™ (BD Science, USA), and data were analysed using the FlowJo software (Tree Star Inc., USA). For each tissue sample, the following gating strategy was used: cells were first distinguished from beads and debris by analysis of forward scatter area (FSC-A) over side scatter area, which were further narrowed down to singlet populations by analysis of FSC-A versus forward FSC height. From these, the 7AAD versus FSC-A profiles were used to distinguish live cells from dead cells. Following this, the total leukocyte population was identified as having a high CD45⁺ profile, which was then subdivided into either a myeloid (CD45⁺CD11b⁺) or lymphoid/non-myeloid (CD45⁺CD11b⁻) cell lineage. From the myeloid lineage, macrophages/microglia were identified with a CD45⁺CD11b⁺F4/80⁺ profile, from which “M2” polarised cells were classified as being CD45⁺CD11b⁺F4/80⁺CD206⁺. From the lymphoid lineage, the total T cell population was based on the number of CD45⁺CD11b⁻CD3⁺ events, which were further divided into either helper (CD45⁺CD11b⁻CD3⁺CD4⁺) or cytotoxic (CD45⁺CD11b⁻CD3⁺CD8⁺) T cells. Finally, B cells were distinguished as having a CD45⁺CD11b⁻CD19⁺ profile.

### Example 15

### Effects of Exosome Administration on Stroke Outcomes Over 7 Days

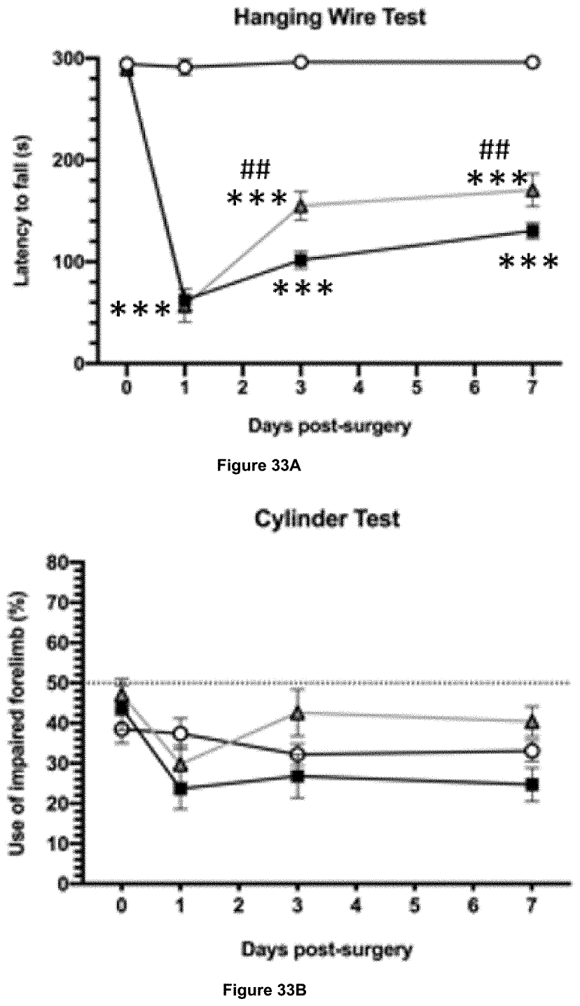

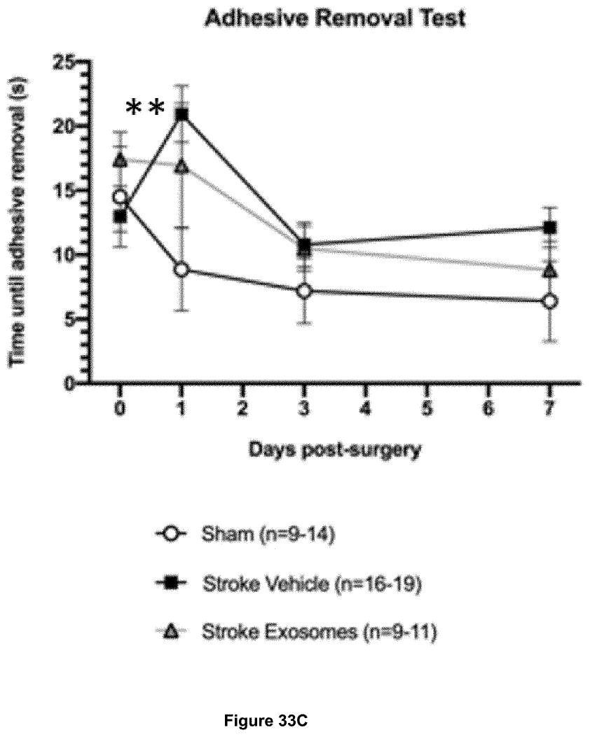

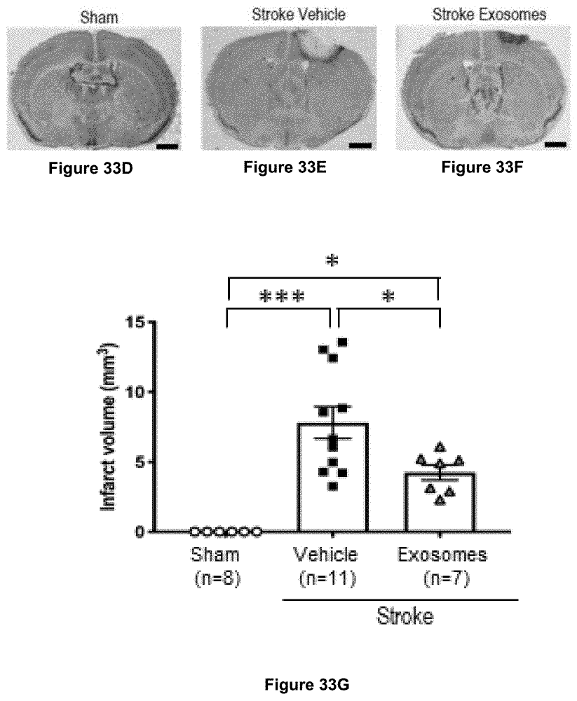

hAEC-derived exosome administration enhances post-stroke recovery in grip function and reduces infarct damage, but does not improve forepaw asymmetry or adhesive removal ability 7 d post-stroke: The hanging wire test was used to assess motor function in terms of grip strength and coordination, and photothrombotic (PT) stroke was found to induce a significant deficit in grip time at 1 day post-stroke (FIG. 33A). Both stroke groups gradually began to recover after this point and despite neither group reaching their original baseline or sham levels by 7 days, mice treated with exosomes displayed a significantly enhanced recovery and were able to hold onto the wire for approximately 53% (P<0.05) and 31% (P<0.05) longer than vehicle-treated mice at day 3 and 7 post-stroke, respectively (FIG. 33A). The cylinder and adhesive removal tests were also conducted to further assess sensorimotor functions, however, exosome administration was not found to enhance recovery in either left (impaired) forepaw use or the ability to remove adhesives at any point over the 7 days (FIGS. 33B-C). Nevertheless, and in conjunction with the improved grip function at 7 d post-stroke, the exosome-treated group exhibited an average infarct volume of 4.2 mm³, which was 46% lower compared to that of the vehicle-treated group at 7 d post-stroke (FIG. 33D, P<0.05).

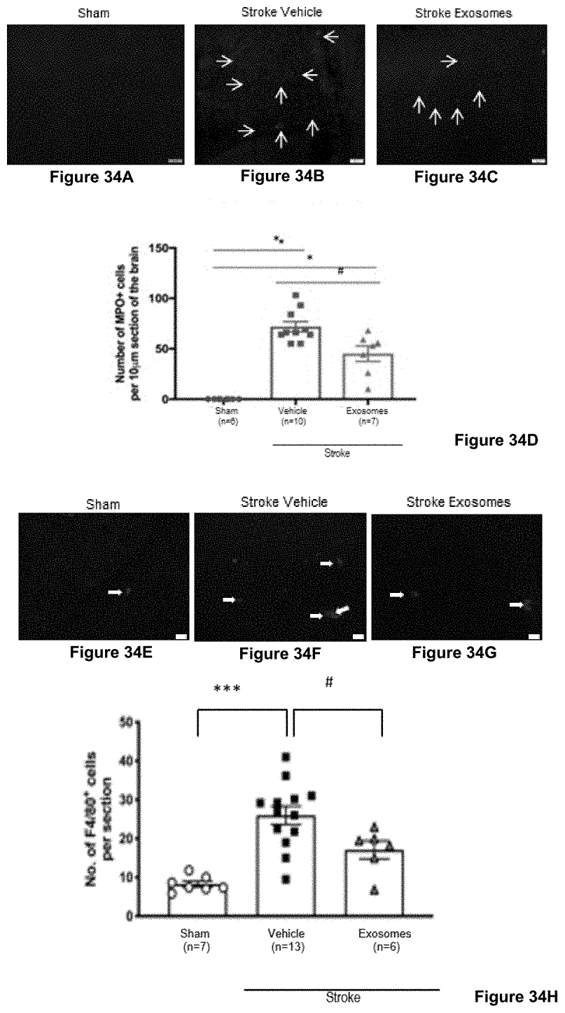

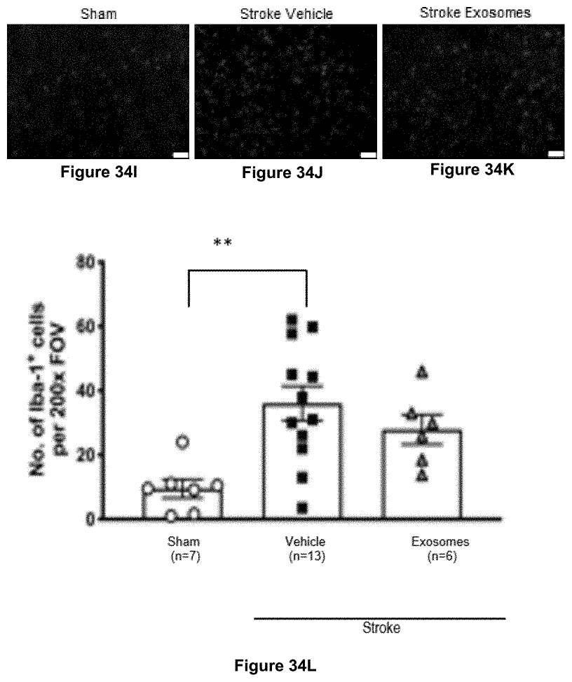

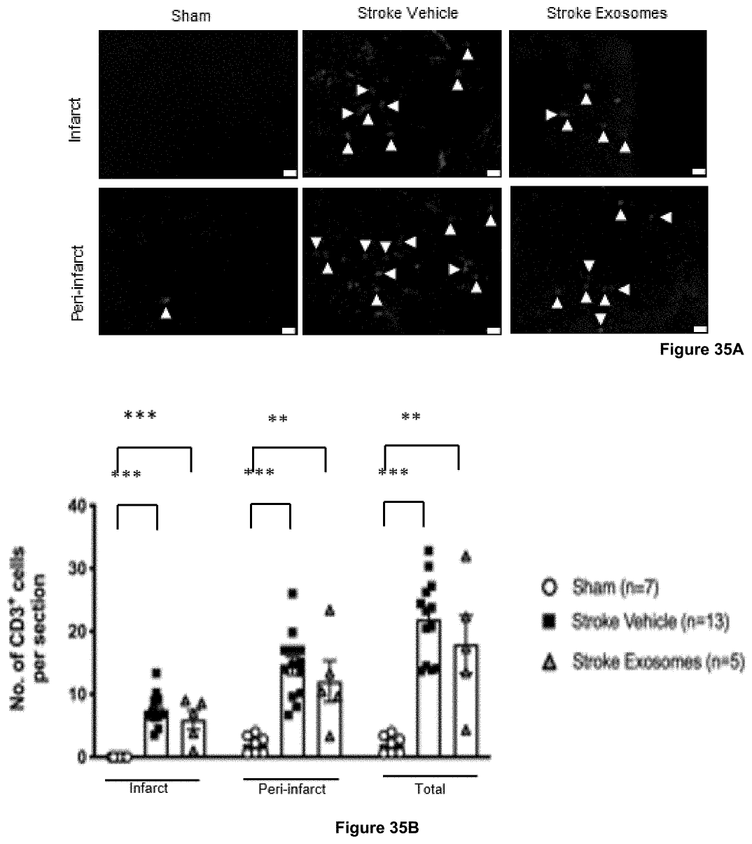

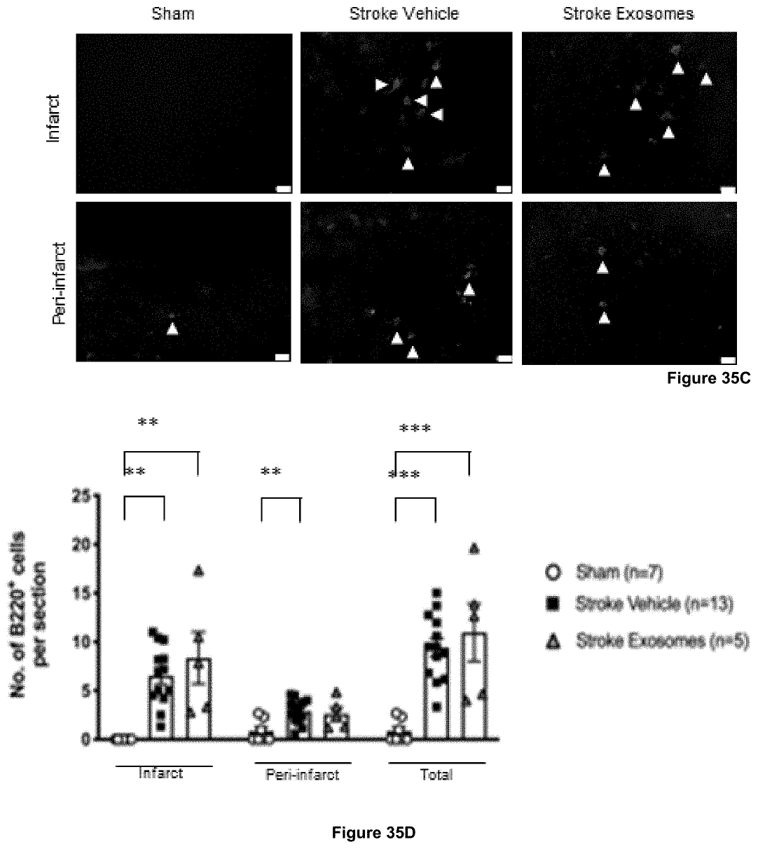

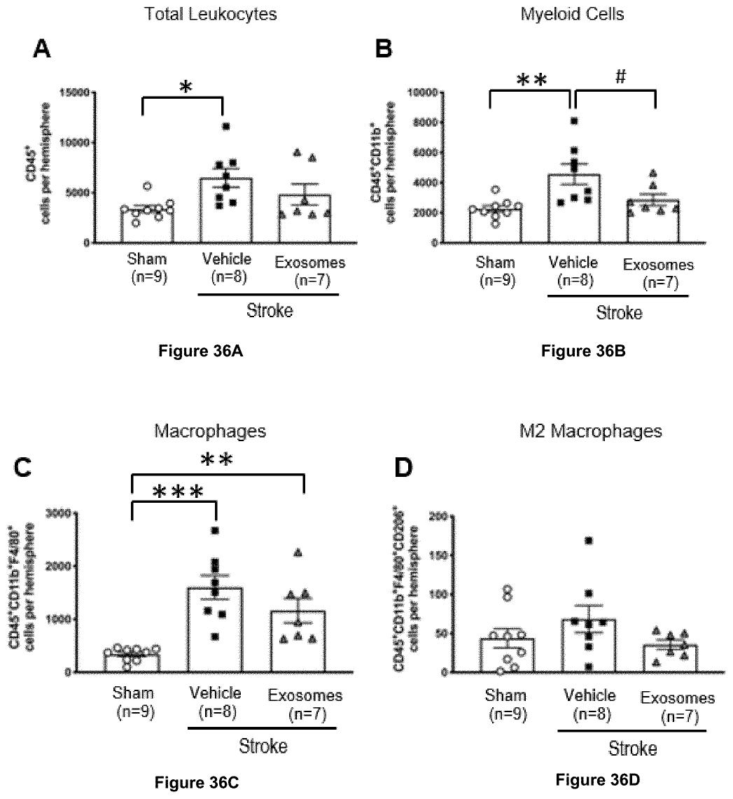

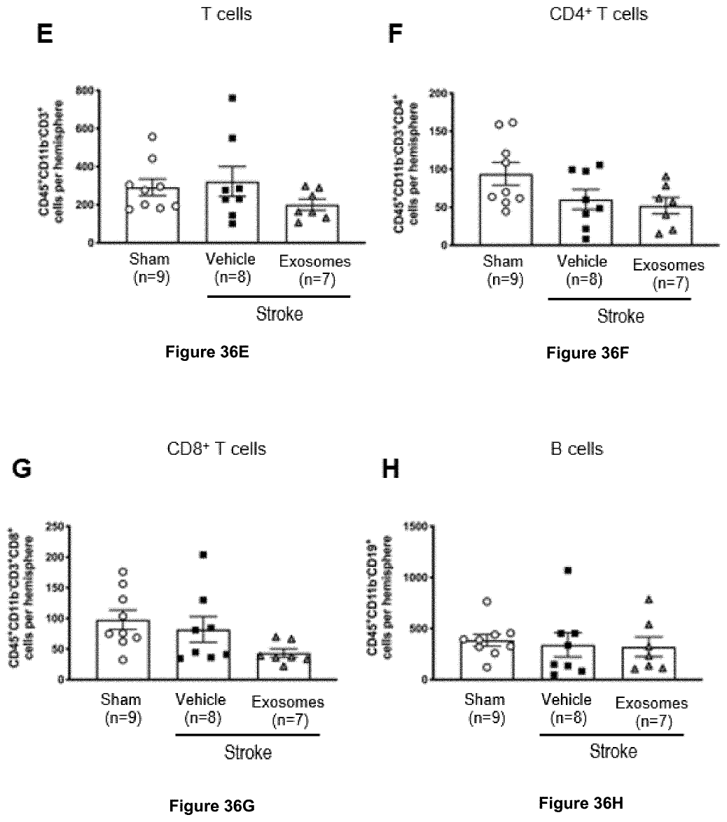

hAEC-derived exosome administration reduces the infiltration of myeloid cells and macrophages, but not microglia or lymphoid cells, into the brain 7 d post-stroke: The effects of hAEC-derived exosomes on the post-stroke inflammatory response were assessed, as inflammation contributes to a considerable portion of brain tissue injury and cell death, particularly in the several days to week following a stroke. Immunofluorescence was used to visualise and semi-quantitatively analyse the presence of immune cells in the brain 7 d post-surgery. PT stroke resulted in a significant increase in the number of F4/80⁺ macrophages, Iba-1⁺ microglia, CD3⁺ T cells and B220⁺B cells surrounding the within the right/ischaemic hemisphere compared to sham, as well as the presence of T and B cells within the infarct core (FIGS. 34A-C, 3A-B; P<0.05 vs sham). Exosome treatment was found to significantly attenuate neutrophil and macrophage infiltration into the ischaemic hemisphere by 40% and 34% (FIGS. 34AB, P<0.05). However, exosome treatment had no effect on microglia, T or B cells (FIGS. 34C, 35A-B). Additionally, flow cytometry was used to provide further quantitative information regarding the effect of exosome treatment on post-stroke inflammation, which revealed stroke-induced exacerbations in the number of total leukocytes, myeloid cells and macrophages compared to sham brains 7 d post-stroke (FIGS. 36A-C, P<0.05). Exosome administration resulted in trends for reductions in total leukocytes and macrophages, and it was found to significantly attenuate the stroke-induced increase in myeloid cells, which include innate immune cells like macrophages (FIG. 36B, P<0.05).

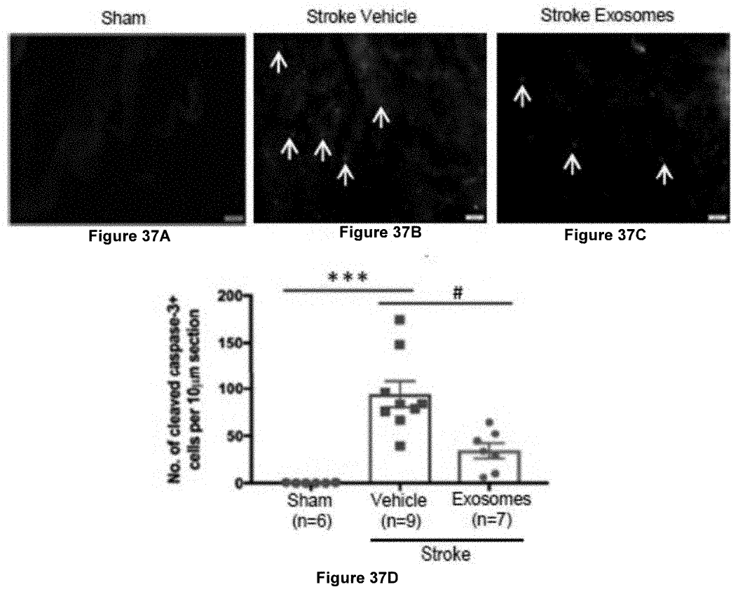

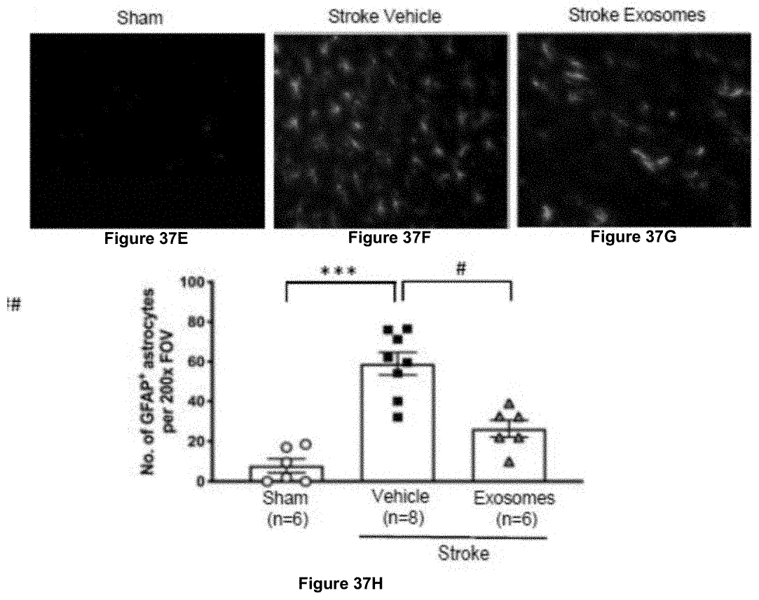

hAEC-derived exosome administration reduces apoptosis and glial scar density 7 d post-stroke. Mice subjected to a PT stroke had a significantly higher number of cleaved-caspase 3+ cells within the ischemic region at 7 days after stroke compared to the sham mice (FIG. 37A, p<0.001). Treatment with exosomes significantly reduced the number of apoptotic cells within the ischemic hemisphere by approximately 60% compared to the vehicle treated group 7 d post-stroke (FIG. 37A, p<0.05).

Cerebral ischaemia also activates astrocytes and increases their expression of the key marker, glial fibrillary acidic protein (GFAP); activated astrocytes contribute to neuronal injury (Choudhury and Ding, 2016, Neurobiology of Disease, 85:234-244; and Yun et al., 2018 Nat Med.;24(7):931-938). PT stroke induced an accumulation of GFAP⁺ astrocytes within the peri-infarct region, forming a glial scar (FIG. 37B). Interestingly, exosome treatment was found to significantly reduce the number of GFAP⁺ astrocytes within the scar by approximately 55% compared to vehicle treatment (FIG. 37B, P<0.05).

### Example 16

### Effects of Exosome Administration on Stroke Outcomes Over 28 Days

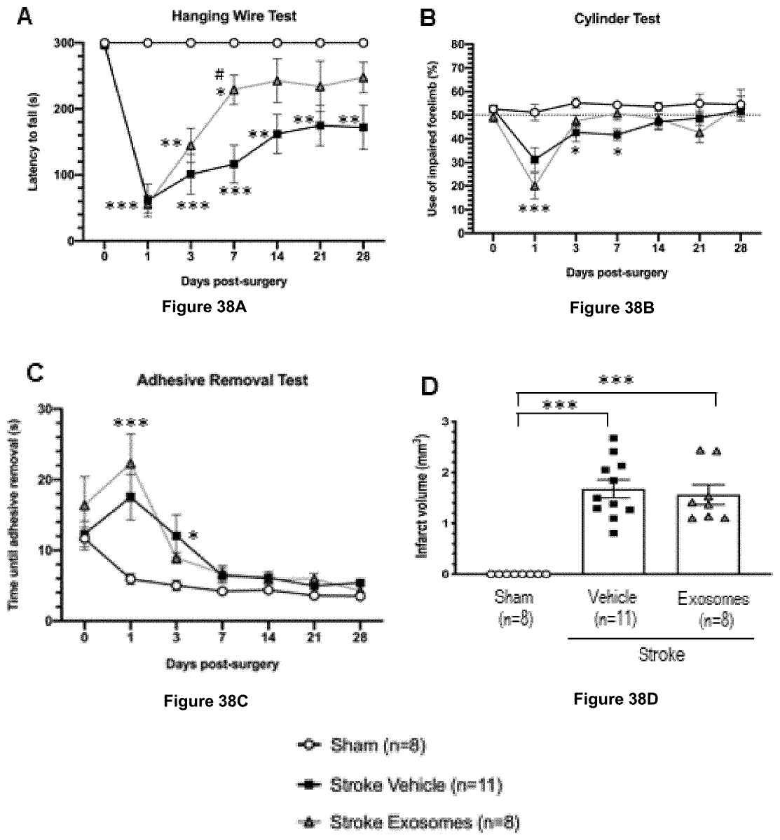

Delayed hAEC-derived exosome administration limits stroke-induced deficits in grip function, but does not improve forepaw asymmetry, adhesive removal or infarct damage 28 d post-stroke: Similar to the first study, mice subjected to PT stroke exhibited sensorimotor deficits across all functional tests at 24 h following stroke induction. In the hanging wire test, whilst all stroke mice began to improve after 24 h, exosome-treated mice displayed greater improvement and a significantly longer grip time on day 7 compared to vehicle (FIG. 38A, P<0.05), and continued to exhibit longer grip times throughout the remaining 3 weeks, however, significance between these groups was lost after day 7. In contrast, exosome and vehicle-treated groups had similar improvements in their ability to remove adhesives and use their left forepaw in the cylinder test, with no significant differences between them at any time-point (FIGS. 38B-C). Furthermore, there was no significant difference in infarct volume between the exosome and vehicle-treated groups at 28 d post-stroke, with both groups exhibiting average volumes between 1.6-1.7±0.18-0.20 mm³ (FIG. 38D).

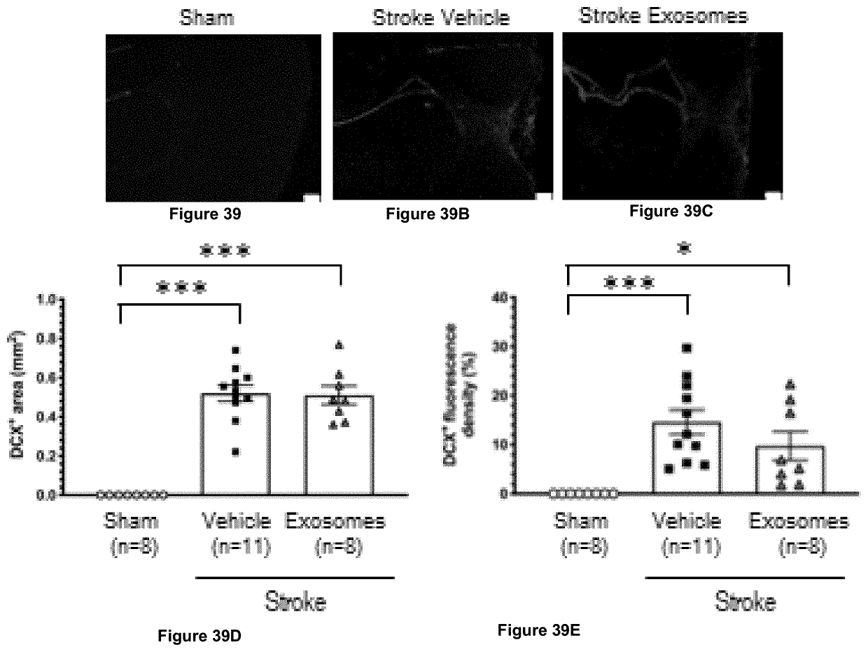

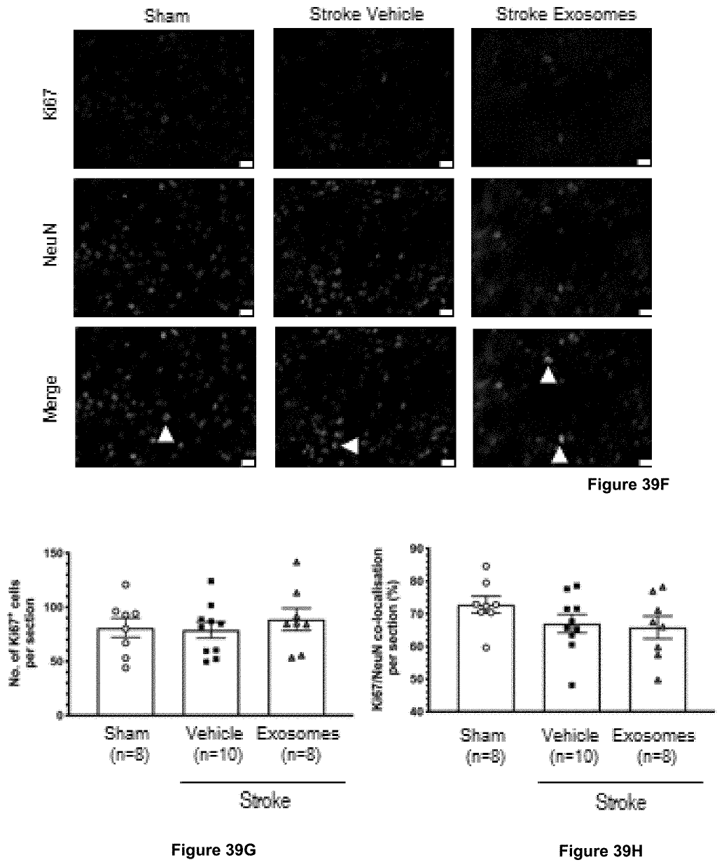

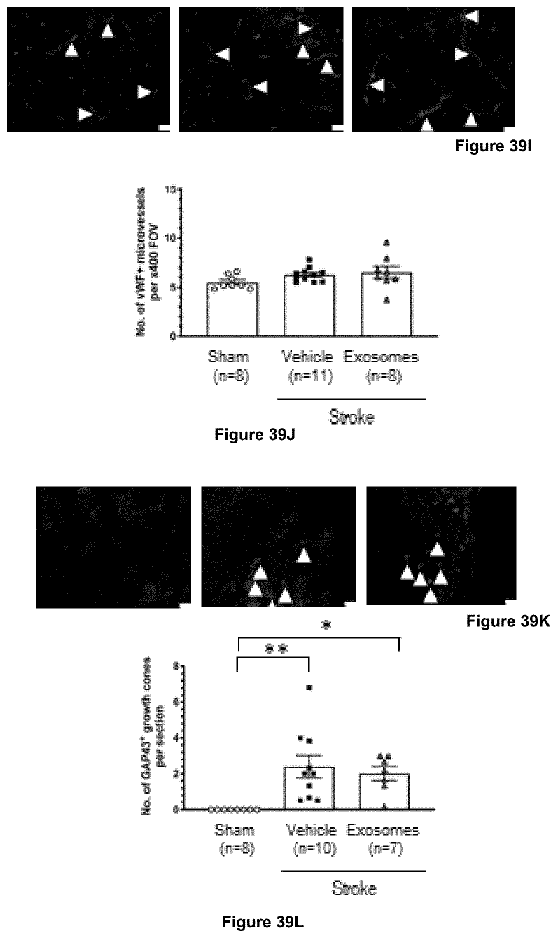

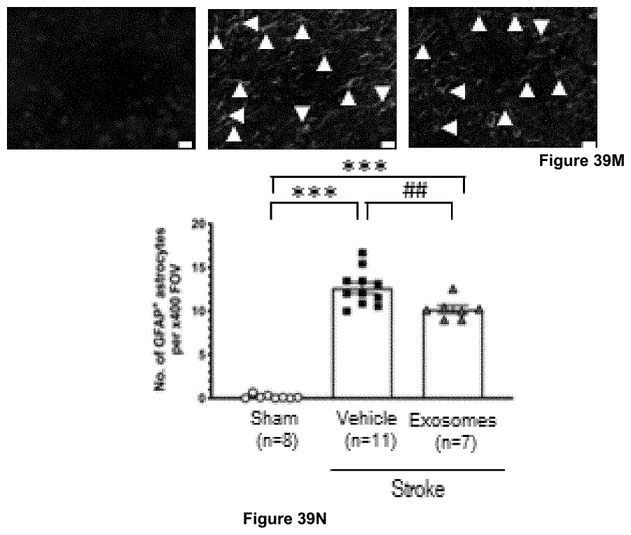

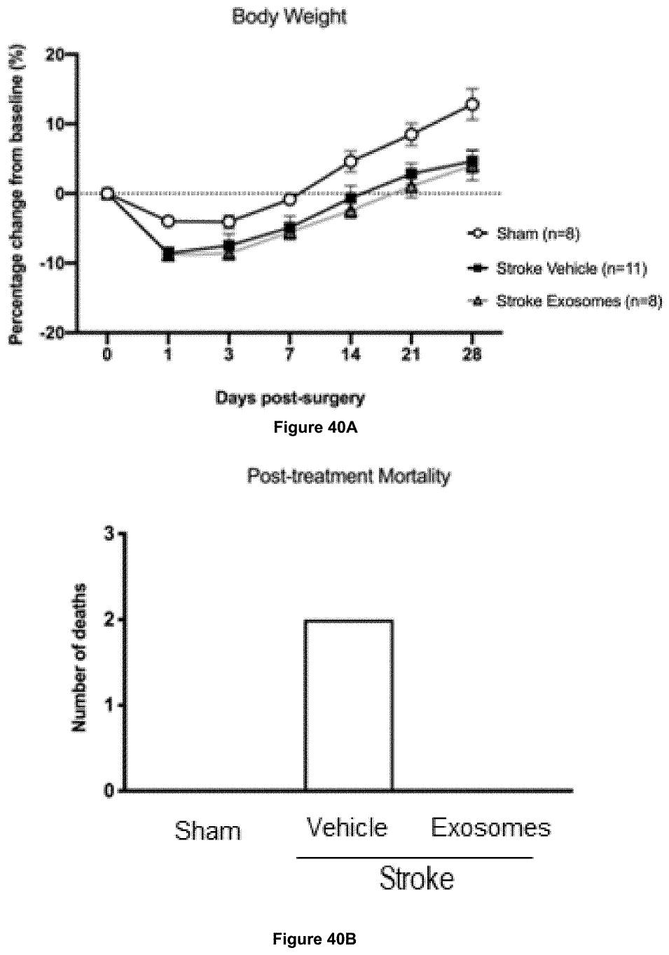

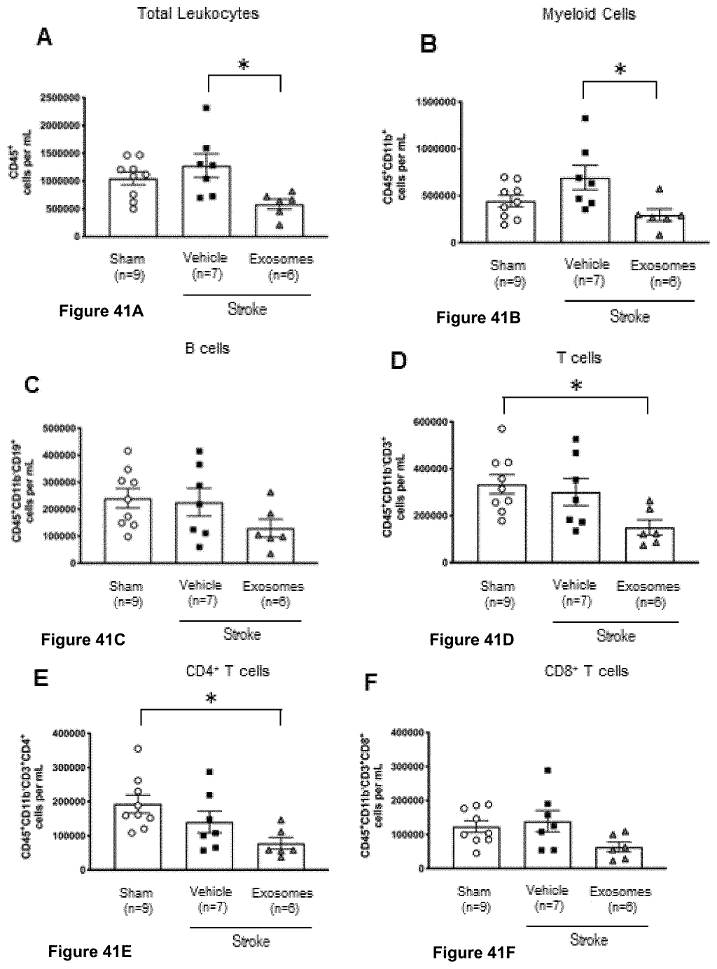

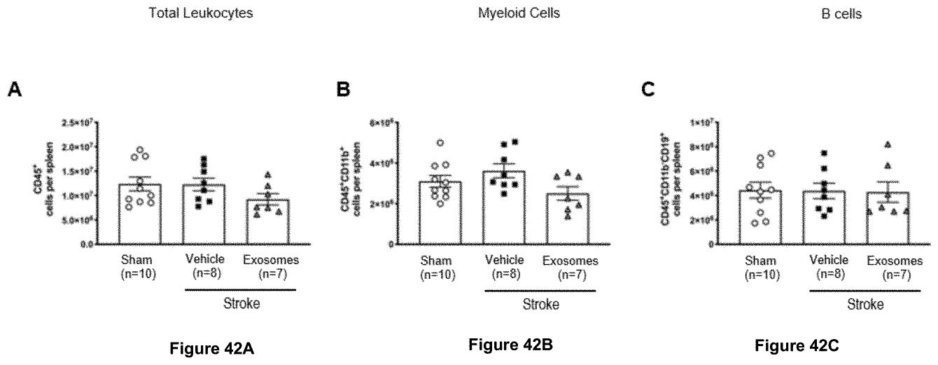

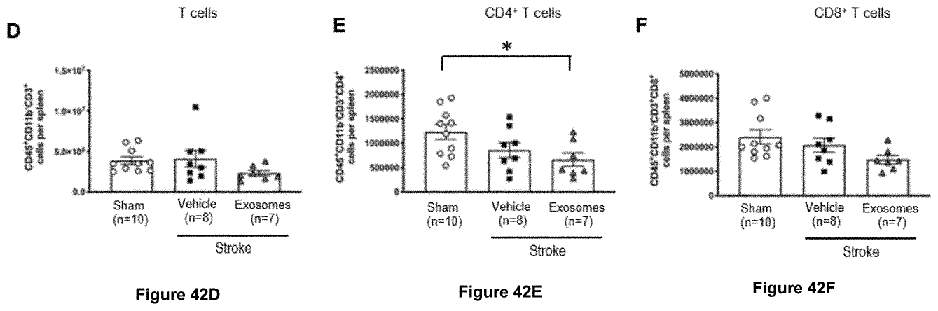

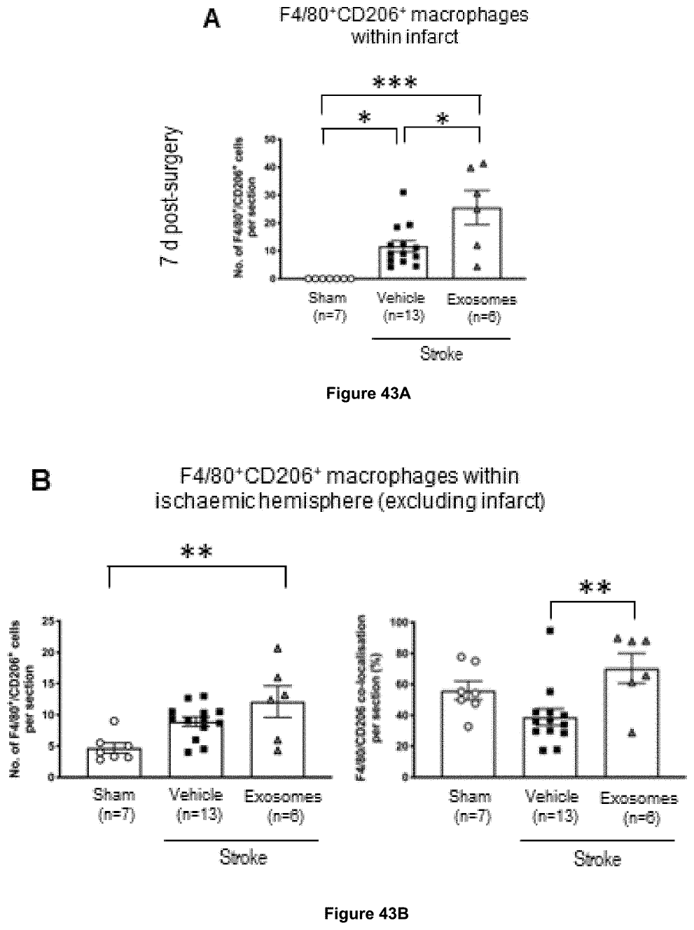

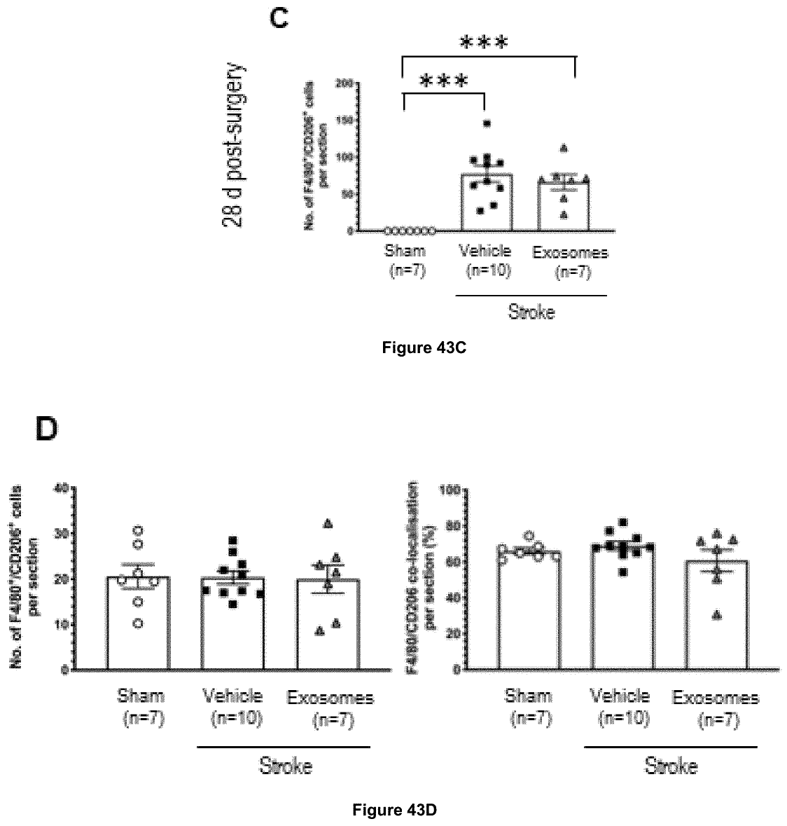

Delayed hAEC-derived exosome administration provides no further enhancement to stroke-induced regenerative mechanisms but reduces glial scar density 28 d post-stroke: It is now well-established that, following initial events of stroke-induced injury mechanisms, the brain undergoes spontaneous repair and regeneration several days to weeks post-stroke (Cramer, 2008, Ann Neurol.; 63(3):272-87 and 63(5):549-560). One such reparative mechanism includes neurogenesis, involving the migration of immature neurons/neuroblasts from the subventricular zone within the lateral ventricles towards the injury site; this can be assessed by immunohistochemical analysis of doublecortin (DCX), a microtubule-associated protein specifically expressed in neuroblasts (Arvidsson et al., 2002, Nat Med.; 8(9):963-70, and Gleeson et al., 1999; Neuron; 23(2):257-71). Indeed, a band of DCX⁺ cells was observed stretching from the right lateral ventricle to the infarct boundary 28 d after PT stroke, however, exosome treatment did not enhance the DCX⁺ immunoreactive area nor fluorescence density (FIG. 39A).

Neuroblasts can proliferate and differentiate into mature neurons, which can be identified using the marker neuronal nuclei (NeuN) alongside the proliferative marker, Ki67. However, there was neither a stroke-induced nor treatment-induced effect on the number of Ki67/NeuN double-positive cells within the ischaemic hemisphere 28 d post-PT stroke (FIG. 39B). In addition, angiogenesis, involving the proliferation of endothelial cells and the formation of new blood vessels, represents another important reparative mechanism occurring after stroke, and can be identified by the expression of von Willebrand factor (vWF), an endothelial cell activation marker. It was found that neither PT stroke nor exosome treatment had any effect on the number of vWF⁺ microvessels within the peri-infarct region when assessed 28 d post-stroke (FIG. 39C).

Stroke is also known to induce endogenous synaptic plasticity, which includes the sprouting and growth of new axons and can be identified by the expression of growth-associated protein-43 (GAP-43), an axonal growth-promoting factor expressed in axonal growth cones. Immunohistochemical analyses revealed the presence of several GAP-43⁺ growth cones within the infarct core post-PT stroke, a result which was absent in sham brain sections, however, exosome treatment provided no further enhancement to the number of GAP-43⁺ growth cones 28 d post-stroke (FIG. 39D).

Finally, similar to the results obtained at 7 d, the glial scar surrounding the infarct remained at 28 d post-stroke, but the density of GFAP⁺ astrocyte within the glial scar was significantly attenuated following exosome administration by approximately 20% compared to vehicle (FIG. 39E, P<0.05).

### Example 17

### Effects of Human Amniotic Epithelial Derived EVs on Demyelination in Cuprizone-Induced Demyelination Model

Multiple sclerosis (MS) is a demyelinating disease of the central nervous system. An investigation was undertaken to study the effect of intranasally applied hAEC derived exosomes (also referred to herein as extracellular vesicles; EV) on Cuprizone induced demyelination, an animal model of MS that allows assessing remyelination without the effect of the peripheral immune system.

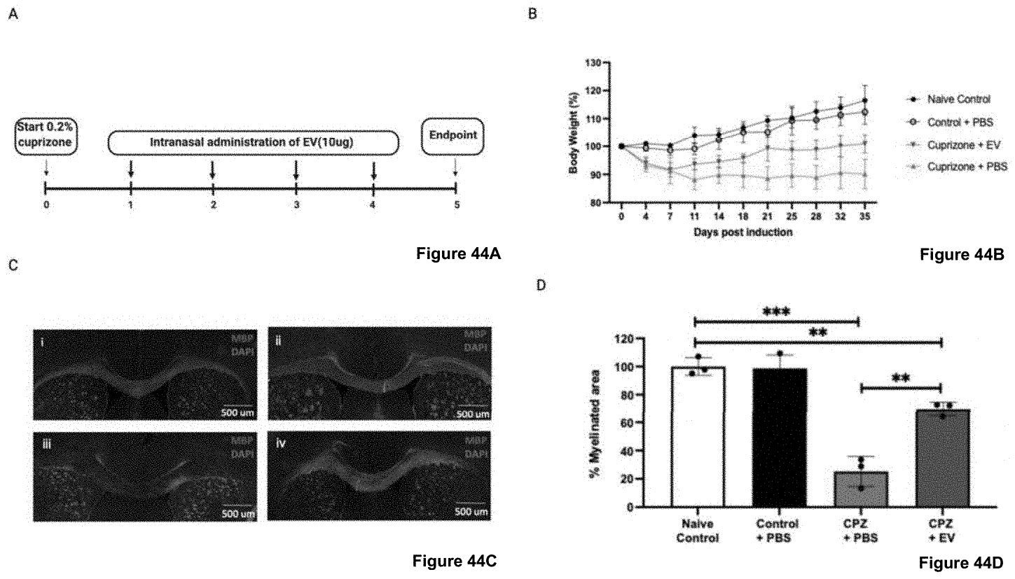

Young adult (8 weeks old) C57Bl/6 mice were subjected to cuprizone-induced demyelination for 5 weeks (FIG. 44) during which the mice were administered EVs intranasally once a week (FIG. 44A). At the end of week 5, mice were culled, and coronal sections were cut from frozen post-mortem brain tissue using cryostat and stained with FluoroMyelin™ Red Fluorescent Myelin and DAPI. The VS120 slide scanner and ImageJ software were used for image acquisition and analysis.

The relative changes in the body weight percentage of mice demonstrated a decrease in the cuprizone-challenged group compared to the control. The group receiving hAEC-EVs showed a weight recovery after EV administration (FIG. 44B). The results of the in vivo study showed that cuprizone-induced demyelination was significantly reduced in the corpus callosum of mice treated with hAEC derived EVs compared with cuprizone-challenged group receiving PBS (FIG. 44D). These findings suggest that intranasal administration of hAEC derived EVs can effectively reduce demyelination in the cuprizone model of multiple sclerosis. Further analysis of the number and activation state of microglia, astrocytes and oligodendrocytes in the brain will provide more information about the therapeutic potential of hAEC derived EVs.

Those skilled in the art will appreciate that the disclosure described herein is susceptible to variations and modifications other than those specifically described. It is to be understood that the disclosure contemplates all such variations and modifications. The disclosure also enables all of the steps, features, compositions and compounds referred to or indicated in this specification, individually or collectively, and any and all combinations of any two or more of the steps or features or compositions or compounds.

### BIBLIOGRAPHY

Anderson et al. (2016) Stem Cells. http://doi.org/10.1002/stem.2298
Doyle et al. (2006) Paediatrics 118: 108-113
Egleton and Davis (1997) Peptides 18:1431-1439
Fishback et al. (2013) Sci Transl Med 5:179 ps7)
Hodges et al. (2012) Am J Obstet Gynerol 206: 448e8-448e15
Langer (1990) Science 249:1527-1533
Lee et al. (2014) Cell 156:440-455
Lim et al. (2013) Placenta 34: 486-492
Lodha et al. (2014) PLoS ONE: e90843
Putney and Burke (1998) Nat Biotech 16:153-157
Remington's Pharmaceutical Sciences (1990), 18th Edition, Mack Publishing Company, Easton, Pa.
Moodley et al. (2010) Am J Respir Crit Care Med i:643-651
Murphy et al. (2012) Cell Transplant 21:1477-1492
Nold et al Proc. Natl Acad. Sci USA 110:14384-14389
Schellenberg et al. (2011) Aging (Albany, N.Y.) 3:873-888
Sayani and Chien (1996) Crit Rev Ther Drug Carrier Syst 13:85-184
Tan et al. (2015) Stem Cell Res Ther 6:8
Vosdoganes et al. (2013) Cytotherapy 15:1021-1029
Yawno et al. (2013) Dev Neurosci 35:272-282

1. A method of treating a mammalian subject, the method comprising the systemic or local administration of mammalian amniotic exosomes derived from allogeneic mammalian amnion epithelial cells derived from a donor mammal of the same species.

2. The method of claim 1, wherein the mammalian subject to be treated is a human.

3. The method of claim 2, wherein the mammalian subject to be treated is a non-human mammal.

4. The method of claim 3, wherein the non-human mammalian subject to be treated is a racing animal selected from the group consisting of a horse, dog, and camel.

5. The method of claim 2, wherein the human subject is treated to induce cellular or neuronal repair, regeneration, or reparation of the central nervous systems, peripheral nervous system, or the systemic vasculature, or for wound healing.

6. The method of claim 5, wherein the human subject is being treated to repair, regenerate, or reparate: (i) cells or tissues of the lungs, heart, liver, kidney, or pancreas; (ii) cells following ischemic-reperfusion injury; (iii) a wound; (iv) brain or spinal cord injury; or (v) to suppress collagen production in activated fibroblasts.

7. The method of claim 6, wherein the human subject is being treated for a fibrotic disease, or for a condition or disorder of the lung, liver, heart, kidney or pancreas.

8. The method of claim 5, wherein the human subject is being treated for neurodegenerative disease or condition.

9. The method of claim 8, wherein the neurodegenerative disease or condition is a demyelination disease.

10. The method of claim 9, wherein the demyleination disease is multiple sclerosis, optic neuritis, Devic's disease, transverse myelitis, acute disseminated encephalomyelitis, adrenoleukodystrophy, or adrenomyeloneuropathy.

11. The method of claim 8, wherein neurodegenerative disease or condition is selected from the group consisting of motor neuron disease, a stroke, spinal cord injury, traumatic brain injury, Alzheimer's disease, Parkinson's disease, Huntington's diseases, and multiple sclerosis.

12. The method of claim 8, wherein the human subject is being treated for a disease or condition selected from the group consisting of bronchopulmonary dysplasia, cystic fibrosis, lung fibrosis, liver fibrosis, chronic lung infection, asthma, allergic rhinitis, and chronic obstructive pulmonary disease (COPD).

13. The method of claim 1, wherein the amniotic exosomes reverse lung infection and fibrosis, and reverse primary lung fibroblasts.

14. The method of claim 1, wherein the amniotic exosomes contain miRNAs which target cytokine-cytokine receptor, Wnt, PI3K-Akt, and TGFβ signaling pathways.

15. The method of claim 4, wherein the racing animal is being treated for exercise induced pulmonary hemorrhage.

16. The method of claim 1, wherein the amniotic exosomes are derived from a bank of immortalized mammalian amnion epithelial cell lines.

17. The method of claim 1, wherein the amniotic exosomes are selected from a bank of lyophilized amniotic exosomes derived from immortalized mammalian amniotic exosomes.

18. A pharmaceutical composition comprising mammalian amniotic exosomes and one or more pharmaceutically acceptable carriers, excipients, and/or diluents.

19. The pharmaceutical composition of claim 18 wherein the mammalian amniotic exosomes are human amniotic exosomes.

20. An improved cell-based therapeutic method for treating a mammalian subject by the use of amnion epithelial cells, the method comprising:
isolating amniotic exosomes from an immortalized amnion epithelial cell line; and
systemically or locally administrating to the subject in need of tissue or neuronal repair, regeneration, and/or reparation.
